(19) United States
(12) Patent Application Publication
(10) Pub. No.: US 2024/0091742 A1
(43) Pub. Date: Mar. 21, 2024

(54) COMPONENTS FOR SEPARATING MOLECULES AND METHODS OF MAKING AND USING THE SAME

(71) Applicant: Pierce Biotechnology Inc., Rockford, IL (US)

(72) Inventors: Ramesh Ganapathy, Rockford, IL (US); Brendon Wang, South Beloit, IL (US); Ashok Salunkhe, Rockton, IL (US); Deven Etnyre, Poplar Grove, IL (US); Barbara Kaboord, Oregon, WI (US)

(21) Appl. No.: 18/229,553

(22) Filed: Aug. 2, 2023

Related U.S. Application Data
(60) Provisional application No. 63/524,189, filed on Jun. 29, 2023.
(60) Provisional application No. 63/370,277, filed on Aug. 3, 2022.

(51) Int. Cl. B01J 20/288; C07K 16/06; B01D 15/34; B01D 15/38; B01J 20/24; B01J 20/26; B01J 20/30; B01J 20/32

(52) U.S. Cl. CPC B01J 20/288; C07K 16/065; B01D 15/34; B01D 15/3847; B01J 20/24; B01J 20/267; B01J 20/3085; B01J 20/3212; B01J 20/3293; B01J 20/3219; B01J 20/3282; B01J 20/3248; B01J 2220/54

(57) ABSTRACT

Disclosed herein are embodiments of matrixes made of a porous size exclusion support and a cationic moiety for separating one or more small molecules from one or more large molecules in a sample using differences in one or more properties such as the size of the molecules, charge of the molecules, the isoelectric point (pI) of the molecules, and/or any combination of these properties including methods, systems, and kit embodiments. Also disclosed herein are embodiments of a method of making the matrixes and using the matrixes for separating small molecules from one or more large molecules in a sample.

### CROSS REFERENCE TO RELATED APPLICATIONS

This application claims the benefit of, and priority to, U.S. Provisional Application No. 63/524,189, filed Jun. 29, 2023, and U.S. Provisional Application No. 63/370,277, filed on Aug. 3, 2022; each of these prior applications is incorporated herein by reference in its entirety.

### FIELD

The present disclosure concerns a matrix, system, method, and kit for sample preparation, such as separating small molecules from large molecules.

### BACKGROUND

Sample preparation techniques for isolating biomolecules may require separating biomolecules from other sample components and from sample processing components to enable downstream analysis and processing of the biomolecule. During sample preparation of biomolecules, such as proteins or nucleic acids, there often is a need to label the biomolecules with dyes, affinity tags, radioactive labels, mass tags, and the like. In other instances, the biomolecules may need to be chemically modified, such as by reduction, oxidization, cross-linking, and/or alkylation.

One example is provided by considering Bovine Serum Albumin (BSA), which is a protein isolated from bovine blood plasma that is commonly added as a stabilizing agent to antibodies. While BSA has stabilizing properties, it often interferes with downstream applications (e.g., labeling of antibodies with fluorospheres), especially in the presence of low antibody levels. To avoid such interference, BSA should be separated from the antibody of choice before labeling the antibody. Often such separations are performed using size exclusion resins, which involve cumbersome fast protein liquid chromatography (FPLC) set up purification resins; however, existing resin kits for BSA removal are often ineffective, have low antibody recovery, and/or have low BSA removal properties.

Thus, there is need for an improved purification resin, method, and kit for purifying proteins from samples, whereby the proteins are sufficiently pure for high quality downstream analysis.

### SUMMARY

Disclosed embodiments of the present disclosure advantageously provide superior separation of molecules, such as biomolecules, from each other. In some aspects of the present disclosure, molecules are separated from each other using differences in one or more properties, such as, but not limited to, the size of the molecules, charge of the molecules, the isoelectric point (pI) of molecules, and/or any combination of these properties. Additionally, molecules can be separated from each other based on one or more separation matrix properties, such as, but not limited to, the charge and size exclusion properties. This can advantageously reduce time and expense related to the separating of small molecules from larger molecules. Molecules separated as set forth herein facilitate downstream processing relative to known processes.

In some embodiments, the porous size exclusion support is produced by using sufficient HEC to produce a resin with a molecular weight cut-off (or “MWCO”) greater than or equal to 40 kDa. For example, some embodiments may comprise from 60 grams HEC to 150 grams HEC. In some aspects, 60 grams to 130 grains HEC in a 5 liter reaction scale is used to produce the size exclusion support. In particular aspects disclosed herein, 0.5 liters to 2 liters of resin bed. comprising the porous size exclusion support, is produced from 60 grams FIEC to 130 grams HEC. In some embodiments, the porous size exclusion support may further comprise a crosslinked porous size exclusion support. In some embodiments, the porous size exclusion support can be crosslinked with an epoxide-containing compound comprising at least one epoxide functional group, with exemplary embodiments being crosslinked with epichlorohydrin (also referred to herein as “Epi”). For example, the porous size exclusion support can be crosslinked with 250 milliliters Epi to 450 milliliters Epi.

Certain disclosed embodiments concern a matrix comprising a porous size exclusion support having a molecular weight cutoff of greater than or equal to 40 kDa, and at least one cationic moiety associated with the porous size exclusion support, wherein the at least one cationic moiety is selected for association with a small molecule having a molecular weight less than 100 kDa, such as, but not limited to, Bovine Serum Albumin (BSA), and/or other proteins. In some embodiments, at least one cationic moiety associates with the small molecule by ionic interaction, hydrophilic interactions, hydrophobic interactions, affinity interaction, hydrogen bonding, and/or van der Waals forces. The cationic moiety may be, for example, an amine, a diamine, a polyamine, amine-containing heterocyclic compounds, amine-containing aromatic compound, or an amine-containing polymer. Particular examples of cationic moieties include 5,8-dimethyl-4,7,10-trioxatridecane-2,12-diamine, polyethylene imine, diaminopentane, N,N-diethylethylenediamine, 1,2-diaminobenzene, 1,3-diaminobenzene, 1,4-diaminobenzene, and (S)-N-boc -2,3 -epoxypropylamine. For certain embodiments, the cationic moiety is covalently bound to the porous size exclusion support.

The matrix may be advantageously equilibrated with an equilibration buffer, comprising little to no salt (e.g., NaCl and other ionic salts), such as from 0 mM of salt to 5 mM of salt. In some embodiments, the equilibration buffer may comprise a positive charge. In another embodiment, the equilibration buffer may comprise a neutral charge. In some aspects, the equilibration buffer can have a pH of 4 to a pH of 9, preferably a pH of 5 to pH of 7. In some aspects, the equilibration buffer can have a concentration of from 20 mM to 100 mM. In particular aspects disclosed herein, the equilibration buffer may comprise triethylammonium bicarbonate, borate, sodium acetate, or HEPES to facilitate separating a small molecule from a larger molecule. For example, separating a negatively charged small molecule from a positively charged large molecule.

The present disclosure, in some embodiments, also provides a system for separating molecules of varying molecular weights and/or charges and/or isoelectric point (pI) values from each other. In one embodiment, a system of the disclosure can separate small molecule from a large molecule in a sample. In one embodiment, a system can of the disclosure can separate small molecules, such as proteins of 70 kDa, from large molecules, such as proteins of 150 kDa, from a sample. Such systems comprise a container comprising a matrix of the present disclosure, and a receptacle located to receive flow from the container. The system may be configured for gravity flow operation, centrifugal force operation, positive pressure operation, negative pressure operation, vacuum operation, or combinations thereof. The container may be any suitable container, such as a columnar container, a tube, a multi-well tube, a multi-well plate, or a multi-well filter plate.

A method for making a matrix comprising a porous size exclusion support having a MWCO of greater than or equal to 40 kDa, and at least one cationic moiety associated with the porous size exclusion support, is also disclosed. The method comprises providing a porous size exclusion support comprising hydroxyethyl cellulose having a MWCO of greater than or equal to 40 kDa or greater, the hydroxyethyl cellulose having at least one vicinal diol. The vicinal diol is oxidized to form an aldehyde, and the aldehyde is then reacted with an amine group of a cationic moiety of at least greater than 50 mg/mL via reductive amination using, for example, sodium cyanoborohydride or picoline borane. The concentration of the cationic moiety that is used to form the resin can be varied, as desired, to facilitate separation processes, but typically is within a concentration of 50 mg/mL to 175 mg/mL, more typically 50 mg/mL to 160 mg/mL.

A method for separating small molecules of varying molecular weights and/or charges and/or pI values from each other is also disclosed. In one embodiment, a method of the disclosure can separate a protein of less than or equal to 70 kDa from at least one large molecule of greater than 100 kDa in a sample. The method comprises providing a matrix comprising a porous size exclusion support having at least one cationic moiety associated therewith, wherein the cationic moiety can associate with at least one small molecule, such as but not limited to, a negatively charged small molecule. In some embodiments, the matrix can be equilibrated with an equilibration buffer. In some aspects of the disclosure, a sample is then applied to the matrix to separate the at least one small molecule from the at least one large molecule by subjecting the matrix to gravity flow, a centrifugal force, a positive pressure, a negative pressure, a vacuum, or a combination thereof. At least one large molecule in the sample is excluded by the matrix and is collected as a flow-through in a receptacle located to receive the flow-through. The at least one small molecule (e.g., a negatively charge small molecule) associates with the at least one cationic moiety and is thereby separated from the at least one large molecule in a single step. The disclosed method substantially increases the ability to separate small molecules, such as Bovine Serum Albumin, from the sample, and also substantially increases the recovery of the large molecule, such as IgG. This in turn facilitates processing the large molecule in downstream applications, such as dye labeling.

A kit for separating molecules of varying molecular weights and/or charges and/or pI values from each other also is disclosed. In one embodiment, a kit of the disclosure can separate small molecules, such as proteins of 70 kDa, from large molecules, such as proteins as large as 150 kDa, from a sample. In another embodiment, the kit of the disclosure can separate negatively charged molecules from more positively charged molecules in a sample. In some embodiments, the kit may comprise (1) a porous size exclusion support having at least one cationic moiety associated therewith, wherein the cationic moiety can associate with and capture the small molecule, and (2) instructions for using the porous size exclusion support. The kit may further comprise an equilibration buffer, and/or a system comprising a container housing the porous size exclusion support, and a receptacle positioned to receive flow-through the support.

The foregoing and other objects, features, and advantages of the present disclosure will become more apparent from the following detailed description, which proceeds with reference to the accompanying figures.

### BRIEF DESCRIPTION OF THE DRAWINGS

FIG. 1 is a schematic drawing illustrating one disclosed embodiment comprising associating a porous size exclusion support with a cationic moiety, such as diaminopentane (or “PDA”).

FIG. 2 is a schematic drawing illustrating one disclosed embodiment comprising associating a porous size exclusion support with a cationic moiety, such as a branched polyethylene imine (or “PEI”).

FIG. 3 is a schematic drawing illustrating one disclosed embodiment comprising associating a porous size exclusion support with a cationic moiety, such as N,N diethylethylenediamine (or “DEED”).

FIG. 4 is a schematic drawing illustrating one disclosed embodiment of using a cationic moiety associating onto a porous size exclusion support to separate a small molecule from a sample comprising the small molecule such as but not limited to, a negatively charged small molecule.

FIG. 5 is a schematic drawing illustrating one embodiment of a disclosed system comprising a container, receptacle, and an exemplary matrix comprising a porous size exclusion support associated with a cationic moiety.

FIG. 6 is a schematic side view of a one embodiment of a system according to the present disclosure comprising a container and a receptacle for processing a sample to separate at least one small molecule from at least one large molecule using differences in one or more properties, such as but not limited to, the size of the molecules, charge of the molecules, the isoelectric point (pI) of molecules, and/or any combination of these properties.

FIG. 7 is a schematic perspective view of one embodiment of a disclosed system for processing a sample to separate a small molecule from a large molecule using differences in one or more properties, such as but not limited to, the size of the molecules, charge of the molecules, the isoelectric point (pI) of molecules, and/or any combination of these properties, and wherein the system comprises a multi-well container and a receptacle.

FIG. 8 is a schematic perspective view of one embodiment of a disclosed system for processing a sample to separate a small molecule from a large molecule using differences in one or more properties, such as but not limited to, the size of the molecules, charge of the molecules, the isoelectric point (pI) of molecules, and/or any combination of these properties, and wherein the system comprises a multi-well container and a receptacle.

FIG. 9 is an image of a gel comparing the BSA removal and IgG recovery of the following embodiments disclosed herein at Table 2 from a sample comprising a mixture of BSA (2 mg/mL) and IgG (2 mg/mL): Resin A, Resin B, Resin C, Resin D, Resin E, and Resin F; wherein FIG. 9 shows that the greatest to least BSA removal was achieved in the following order (and thus shows that in addition to the PDA, the MWCO also contributed to the desired BSA removal): Resin F, Resin D, Resin E, Resin B, then Resin A.

FIG. 10 is an image of a gel comparing the Resin 5 embodiment (as described in Table 1, provided herein), the Resin L embodiment (as described in Table 2, provided herein), the Resin F embodiment (as described in Table 2, provided herein), and the Resin D embodiment (as described in Table 2, provided herein) were equilibrated with different equilibration buffers (as described in Table 3, provided herein) to remove BSA and recover IgG from a sample comprising a mixture of BSA (10 mg/mL) and IgG (1 mg/mL); thus, this figure demonstrates that PDA helps remove a desirable amount of BSA, the embodiments having a MWCO of greater than or equal to 40 kDa exhibited a lower capacity for removing BSA than the 45 kDa MWCO embodiments, and the embodiments equilibrated with Tris buffer showed a desirable IgG recovery.

FIG. 11 is an image of a gel showing the removal of BSA and recovery of IgG from a

sample comprising a mixture of BSA (10 mg/mL) and IgG (1 mg/mL) by the Resin G embodiment (as described in Table 1, provided herein) equilibrated with 50 mM Tris pH 7 (Lane 1), 50 mM TEAB pH 5 (Lane 2), 50 mM TEAB pH 7 (Lane 3), 50 mM sodium acetate pH 5 (Lane 4), 50 mM sodium acetate pH 7 (Lane 5), 50 mM HEPES pH 5 (Lane 6), 50 mM HEPES pH 7 (Lane 7) showing desirable BSA removal and IgG recovery from the sample.

FIG. 12 is a bar graph comparing the BSA binding capacity (300 μL of 10 mg/mL BSA) of the Resin 5 embodiment (as described in Table 1, provided herein) modified with 150 mg/mL PEI, the Resin 3 embodiment (as described in Table 1, provided herein) modified with 130 mg/mL PEI , Melon™ Gel IgG Purification Kit, and Affi-Gel® Blue Gel (Bio-Rad); the Resin 5 embodiment exhibited a binding capacity of 3.11 mg BSA bound/mL resin, the Resin 3 embodiment exhibited a binding capacity of 2.45 mg BSA bound/mL resin, the Melon™ Gel IgG Purification Kit exhibited a binding capacity of 1.26 mg BSA bound/mL resin, and the Affi-Gel® Blue Gel (Bio-Rad) exhibited a binding capacity of 2.43 mg BSA bound/mL resin; thus, this figure shows a desirable BSA binding capacity in the Resin 5 embodiment modified with 150 mg/mL PEI, the Resin 3 embodiment modified with 150 mg/mL PEI, and the Affi-Gel® Blue Gel (Bio-Rad) unlike the Melon™ Gel IgG Purification Kit.

FIG. 13 is a bar graph comparing the IgG recovery (2 mg/mL) of the Resin 5 embodiment (as described in Table 1, provided herein) modified with 150 mg/mL PEI, the Resin 3 embodiment (as described in Table 1, provided herein) modified with 130 mg/mL PEI, Melon™ Gel IgG Purification Kit, and Affi-Gel® Blue Gel (Bio-Rad); the Resin 5 embodiment exhibited a 72% volume recovery, the Resin 3 embodiment exhibited a 61% volume recovery, the Melon™ Gel IgG Purification Kit exhibited a 80% volume recovery, and the Affi-Gel® Blue Gel (Bio-Rad) exhibited a 25% volume recovery; thus, this example shows a desirable IgG recovery in the Resin 5 embodiment modified with 150 mg/mL PEI, the Resin 3 embodiment modified with 130 mg/mL PEI, and the Melon™ Gel IgG Purification Kit unlike the Affi-Gel® Blue Gel (Bio-Rad).

FIG. 14A is an image of a gel comparing the Resin 5 embodiment (as described in Table 1, provided herein) produced with different amounts of PDA (as described in Table 4, provided herein) to Abcam BSA Removal Kit and the Melon™ Gel IgG Purification Kit to remove BSA and recover IgG from a sample comprising a mixture of BSA (10 mg/mL) and GAR (1 mg/mL); the Resin 5 embodiment modified with 150 mg/mL PDA (also referred to herein as the Resin F embodiment as described in Table 2, provided herein) and the Resin 5 embodiment modified with 75 mg/mL PDA (also referred to herein as the Resin K embodiment as described in Table 2, provided herein) showing higher BSA removal and higher recovery of IgG than Abcam BSA Removal Kit and Melon™ Gel IgG Purification Kit.

FIG. 14B is a bar graph showing the band quantification of the gel of FIG. 14A using iBright Image analysis software demonstrating the Resin 5 embodiment modified with 150 mg/mL PDA had a 85.7% IgG recovery and a 98.8% BSA removal; Resin 5 modified with 75 mg/mL showed an 86.7% IgG recovery and an 82.6% BSA removal; Abcam BSA Removal Kit had a 53.3% IgG recovery and a 65.78% BSA removal; and Melon™ Gel IgG Purification Kit had a 78.7% IgG recovery and a 8.3% BSA removal; thus, demonstrating the Resin 5 modified with 150 mg/mL PDA and 75 mg/mL PDA achieved a higher IgG recovery and BSA removal than the Abcam BSA Removal Kit and the Melon™ Gel IgG Purification Kit.

FIG. 15 is a bar graph showing the percent recovery of different sized molecules to establish a MWCO of the Resin 5 embodiment, the Resin 6 embodiment, the Resin 7 embodiment, and the Resin 8 embodiment (as described in Table 1, provided herein) showing the Resin 5 embodiment exhibited an 86% at 42,000 Da, 94% recovery at 67,000 Da, 92% recovery at 80,000 Da, and 94% recovery at 150,000 Da; the Resin 6 embodiment exhibited an 82% recovery at 42,000 Da, 84% recovery at 67,000 Da, 90% recovery at 80,000 Da, and 92% recovery at 150,000 Da; the Resin 7 embodiment exhibited a 66% recovery at 42,000 Da, 76% recovery at 67,000 Da, 84% recovery at 80,000 Da, and 86% recovery at 150,000 Da; the Resin 8 embodiment exhibited a 58% recovery at 42,000 Da, 75% recovery at 67,000 Da, 75% recovery at 80,000 Da, and 83% recovery at 150,000 Da; thus, this figure demonstrates that by decreasing the amount of HEC, resulted in resins with a 50 kDa MWCO, 80 kDa MWCO, and a 90 kDa MWCO (as described in Table 1, provided herein).

FIG. 16A is an image of a gel showing the removal of BSA and IgG recovery for the Resin F embodiment, the Resin G embodiment, the Resin H embodiment, the Resin I embodiment, and the Resin J embodiment (as described in Table 1, provided herein) versus Melon™ Gel IgG Purification Kit (Thermo Scientific™), Affi-Gel® Blue Gel (Bio-Rad), and Abcam BSA Removal Kit from a sample comprising a mixture of BSA (10 mg/mL) and GAR IgG (1 mg/mL); thus, this figure demonstrates the Resin F embodiment and the Resin G embodiment had the most desirable BSA removal and IgG recovery from the sample.

FIG. 16B a bar graph showing the band quantification of the gel of FIG. 16A using iBright Image analysis software demonstrating the that Resin F embodiment had a 83% GAR recovery and a 99% BSA removal; the Resin G embodiment had a 93% GAR recovery and a 100% BSA removal; the Resin H embodiment had a 63% GAR recovery and a 99% BSA removal; the Resin I embodiment had a 76% GAR recovery and a 95% BSA removal; the Resin J embodiment had a 82% GAR recovery and a 100% BSA removal; Melon™ Gel IgG Purification Kit (Thermo Scientific™) had a 106% GAR recovery and a 82% BSA removal; Affi-Gel® Blue Gel (Bio-Rad) had a 73% GAR recovery and a 65% BSA removal; Abcam BSA Removal Kit had a 125% GAR recovery and a 85% BSA removal; thus, this figure demonstrates higher BSA removal and IgG recovery by the Resin F embodiment, the Resin G embodiment, and the Resin J embodiment.

FIG. 17 is am image of a gel showing the BSA removal and IgG recovery of the Resin F embodiment, the Resin G embodiment, the Resin H embodiment, the Resin I embodiment, and the Resin J embodiment (as described in Table 2, provided herein) versus the Melon™ Gel IgG Purification Kit (Thermo Scientific™), Affi-Gel® Blue Gel (Bio-Rad), and Abcam BSA Removal Kit for a sample comprising a mixture of BSA (10 mg/mL) and IgG (0.1 mg/mL); thus, this figure demonstrates that the Resin F embodiment, the Resin G embodiment, and the Resin J embodiment performed better in mixture having a low concentration of IgG versus the Melon™ Gel IgG Purification Kit (Thermo Scientific™), Affi-Gel® Blue Gel(Bio-Rad), and Abcam BSA Removal Kit.

FIG. 18 is an image of a fluorescently labeled GAR flow-through with NHS DyLight™ 488 (Thermo Scientific™) after removing BSA from a mixture comprising GAR (1 mg/mL) and BSA (10 mg/mL) with the Resin F embodiment (as described in Table 2, provided herein) versus Melon™ Gel IgG Purification Kit (Thermo Scientific™), Affi-Gel® Blue Gel (Bio-Rad), and Abcam BSA Removal Kit; thus, this figure demonstrates the desirable ability to recovery the labeled GAR after the BSA removal by the Resin F embodiment and Abcam BSA Removal Kit unlike Melon™ Gel IgG Purification Kit (Thermo Scientific™) and the Affi-Gel® Blue Gel (Bio-Rad).

FIG. 19 is an image of a fluorescently labeled GAR flow-through with NHS DyLight™ 488 (Thermo Scientific™) after removing BSA from a mixture comprising GAR (0.1 mg/mL) and BSA (10 mg/mL) with the Resin F embodiment and the Resin G embodiment (as described in Table 2, provided herein) versus Melon™ Gel IgG Purification Kit (Thermo Scientific™), Affi-Gel® Blue Gel (Bio-Rad), and Abcam BSA Removal Kit; thus, this figure demonstrates the desirable ability to recover the labeled GAR after the BSA removal by the Resin F embodiment and Resin G embodiment even at low antibody concentrations, such as 0.1 mg/mL unlike Melon™ Gel IgG Purification Kit (Thermo Scientific™), Affi-Gel® Blue Gel (Bio-Rad), and Abcam BSA Removal Kit.

FIG. 20 is an image of a gel showing the cleanup of rabbit serum, mouse serum, human plasma, and human serum by the Resin F embodiment and Resin G embodiment (as described in Table 2, provided herein) and demonstrating desirable removal of albumin and desirable IgG recovery from different serum species.

FIG. 21A is a bar graph showing the rabbit IgG (A 280 amount) for the Resin G embodiment (as described in Table 2, provided herein) equilibrated with different concentrations of Tris buffer showing the A 280 amount of the rabbit IgG of the Resin G embodiment equilibrated with 50 mM Tris (pH 7.0) had a 123 A 280 amount; the Resin G embodiment equilibrated with 50 mM Tris (pH 5.0) had a 124 A 280 amount; the Resin G embodiment equilibrated with 50 m Tris (pH 7.0 +stacker 20 μL) had a 139 A 280 amount; the BSA-rabbit IgG start mixture had a 824 A 280 amount; the Rabbit IgG start had a 133 A 280 amount; and the BSA only had a 634 A 280 amount; thus, the figure demonstrates the desirable recovery of the antibody because the flow-through A 280 amount was similar to the A 280 amount of the rabbit IgG.

FIG. 21B is a bar graph showing the GAR (A 280 amount) for the Resin G embodiment (as described in Table 2, provided herein) equilibrated with different concentrations of Tris buffer showing the A 280 amount of the rabbit IgG of the Resin G embodiment equilibrated with 50 mM Tris (pH 7.0) had a 97 A 280 amount; the Resin G embodiment equilibrated with 50 mM Tris (pH 5.0) had a 101 A 280 amount; the resin G embodiment equilibrated with 50 m Tris (pH 7.0+ stacker 20 μL) had a 99 A 280 amount; the BSA-GAR start mixture had a 834 A 280 amount; the GAR start had a 100 A 280 amount; and the BSA only had a 634 A 280 amount; thus, this figure demonstrates the figure demonstrates the desirable recovery of the antibody because the flow-through A 280 amount was similar to the A 280 amount of the GAR.

FIG. 22 is an image showing the labeling of primary antibody (GAPDH) with fluorescent dye after BSA removal from sample comprising a mixture of antibody (1 mg/mL)-BSA (10 mg/mL) for the Resin G embodiment (as described in Table 2, provided herein) spun at 3,000×G and at 6,000×G, the antibody-BSA and free Dy 650 was also labeled; thus this figure shows a desirable BSA removal for both spin speeds.

FIG. 23 is an image a gel obtained by loading and staining the flow-throughs using Coomassie stain using Pierce Power blotter showing the Calreticulin that is free from BSA after passing through the Resin G embodiment (as described in Table 2, provided herein), the Calreticulin that is conjugated to the DyLight™ 680 (Thermo Scientific™) after BSA removal, and the Calreticulin as received with BSA added as a stabilizer; thus, this figure demonstrates the desirable removal of BSA from the primary antibody Calreticulin before conjugating it with DyLight™ (Thermo Scientific™) 680 by the Resin G embodiment.

FIG. 24A is an image of Western Blot application using fluorescent labeled GAR after BSA cleanup with the Resin H embodiment (as described in Table 2, provided herein) showing the BSA removed from GAR (left) and the BSA not removed from GAR (right) from a sample comprising a mixture of GAR (1 mg/mL) and BSA (10 mg/mL); thus, this figure shows that BSA removed from the GAR before conjugating to Dy 650 showed a much higher intensity than when BSA was not removed from the GAR.

FIG. 24B is a bar graph showing the fluorescence intensity of the removed BSA with the Resin H embodiment (as described in Table 2, provided herein) and the unremoved BSA of a HeLa lysate load for the BSA removed at 10 μg had a fluorescence intensity of 13,000,000 and the unremoved BSA had a fluorescence intensity of 4,000,000; HeLa lysate load for the BSA removed at 5 μg had a fluorescence intensity of 9,000,000 and the unremoved BSA had a fluorescence intensity of 3,800,000; HeLa lysate load for the BSA removed at 2.5 μg had a fluorescence intensity of 7,800,000 and the unremoved BSA had a fluorescence intensity of 3,800,000; and HeLa lysate load for the BSA removed at 1.25 μg had a fluorescence intensity of 4,100,000 and the unremoved BSA had a fluorescence intensity of 1,800,000; thus, this figure demonstrates that BSA removed from the GFAR before conjugating to Dy 650 showed a much higher intensity than when BSA was not removed from the GAR and a three times more fluorescence intensity was observed with GAR Dy 650 with BSA removed.

### DETAILED DESCRIPTION

### I. Abbreviations

BSA: Bovine Serum Albumin

DEED: Diethylethylenediamine

Epi: Epichlorohydrin

HEC: hydroxyethyl cellulose

HEPES: 4-(2-hydroxyethyl)-1-piperazineethanesulfonic acid

IgG: Immunoglobulin G

MWCO: Molecular weight cut-off

PDA: Diaminopentane

PEI: Branched polyethylene imine

TRIS: Tris(hydroxymethyl)aminomethane

### II. Overview of Terms, Ranges, and Definitions

Both the foregoing general description and the following detailed description are exemplary and explanatory only and are not intended to limit the scope of the present disclosure.

As used herein, the use of the singular includes the plural unless specifically stated otherwise. For example, the singular forms “a”, “an” and “the” as used in the specification also include plural aspects unless the context dictates otherwise. Similarly, any singular term used in the specification also means plural or vice versa, unless the context dictates otherwise.

In some examples, values, procedures, or devices may be referred to as “lowest,” “best,” “minimum,” or the like. It will be appreciated that such descriptions are intended to indicate that a selection among many used functional alternatives can be made, and such selections need not be better, smaller, or otherwise preferable to other selections.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting, unless otherwise indicated. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximates unless the word “about” is recited. Furthermore, not all alternatives recited herein are equivalents.

The term “or combinations thereof” as used herein refers to all permutations and combinations of the listed items preceding the term. For example, “A, B, C, or combinations thereof” is intended to include at least: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth.

The term “acyl halide” generally refers to —C(O)X, wherein X is a halogen, such as Br, F, I, or Cl.

The term “alcohol” generally refers to an organic compound including at least one hydroxyl group. Alcohols may be monohydric (including one —OH group), dihydric (including two —OH groups; diols, such as glycols), trihydric (including three —OH; triols, such as glycerol) groups, or polyhydric (including two or more —OH groups; polyols). The organic portion of the alcohol may be aliphatic, cycloaliphatic (alicyclic), heteroaliphatic, cycloheteroaliphatic (heterocyclic), polycyclic, aryl, or heteroaryl, and may be substituted or unsubstituted.

The term “aldehyde” generally refers to generally refers to a carbonyl-bearing functional group having a formula

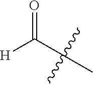 [SMILES: *C=O]

where the line drawn through the bond indicates that the functional group can be attached to any other moiety, but that such moiety simply is not indicated.

The term “aliphatic” generally refers to a substantially hydrocarbon-based compound, or a radical thereof (e.g., C₆H₁₃, for a hexane radical), including alkanes, alkenes, alkynes, including cyclic versions thereof, and further including straight- and branched-chain arrangements, and all stereo and position isomers as well. Unless expressly stated otherwise, an aliphatic group contains from one to twenty-five carbon atoms; for example, from one to fifteen, from one to ten, from one to six, or from one to four carbon atoms. The term “lower aliphatic” refers to an aliphatic group containing from one to ten carbon atoms. An aliphatic chain may be substituted or unsubstituted. Unless expressly referred to as an “unsubstituted aliphatic,” an aliphatic group can either be unsubstituted or substituted. An aliphatic group can be substituted with one or more substituents (up to two substituents for each methylene carbon in an aliphatic chain, or up to one substituent for each carbon of a C═C double bond in an aliphatic chain, or up to one substituent for a carbon of a terminal methine group). Exemplary substituents include, but are not limited to, alkyl, alkenyl, alkynyl, alkoxy, alkylamino, alkylthio, acyl, aldehyde, amide, amino, aminoalkyl, aryl, arylalkyl, carboxyl, cyano, cycloalkyl, dialkylamino, halo, haloaliphatic, heteroaliphatic, heteroaryl, heterocycloaliphatic, hydroxyl, oxo, sulfonamide, sulfhydryl, thioalkoxy, or other functionality.

The term “alkoxy” generally refers to radical (or substituent) having the structure —OR, where R is a substituted or unsubstituted alkyl.

The term “alkyl” generally refers to a hydrocarbon group having a saturated carbon chain. The chain may be cyclic, branched, or unbranched.

The term “alkynyl” generally refers to an organic compound having at least one carbon-carbon triple bond. An alkynyl group can be branched, straight-chain, or cyclic (e.g., cycloalkynyl).

The term “amide” generally refers to chemical functional group —C(O)N(R′)(R″) where R′ and R″ independently hydrogen, alkyl, heteroalkyl, haloalkyl, aliphatic, heteroaliphatic, haloaliphatic, aryl (such as optionally substituted phenyl or benzyl), heteroaryl, haloaryl, alkylsulfano, or other functionality.

The term “amino” generally refers to a chemical functional group —N(R)R′ where R and R′ are independently hydrogen, alkyl, heteroalkyl, haloalkyl, aliphatic, heteroaliphatic, haloaliphatic, aryl (such as optionally substituted phenyl or benzyl), heteroaryl, haloaryl, alkylsulfano, or other functionality.

The term “antibody” generally refers to immunoglobulins or immunoglobulin-like molecules (including by way of example and without limitation, IgA (Immunoglobulin A), IgD (Immunoglobulin D), IgE (Immunoglobulin E), IgG (Immunoglobulin E) and IgM (Immunoglobulin M), combinations thereof, and similar molecules produced during an immune response in any chordate such as a vertebrate, for example, in mammals such as humans, goats, rabbits and mice) and fragments thereof that specifically bind to a molecule of interest (or a group of highly similar molecules of interest) to the substantial exclusion of binding to other molecules. An “antibody” typically comprises a polypeptide ligand having at least a light chain or heavy chain immunoglobulin variable region that specifically recognizes and binds an epitope of an antigen. Immunoglobulins are composed of a heavy and a light chain, each of which has a variable region, termed the variable heavy (V_{H}) region and the variable light (V_{L}) region. Together, the V_{H} region and the V_{L} region are responsible for binding the antigen recognized by the immunoglobulin. Exemplary immunoglobulin fragments include, without limitation, proteolytic immunoglobulin fragments [such as F(ab′)₂ fragments, Fab′ fragments, Fab′-SH fragments and Fab fragments as are known in the art], recombinant immunoglobulin fragments (such as sFv fragments, dsFv fragments, bispecific sFv fragments, bispecific dsFv fragments, ‘F(ab)’₂ fragments, single chain Fv proteins (“scFv”), and disulfide stabilized Fv proteins (“dsFv”). Other examples of antibodies include diabodies, and triabodies (as are known in the art), and camelid antibodies. “Antibody” also includes genetically engineered molecules, such as chimeric antibodies (for example, humanized murine antibodies), and heteroconjugate antibodies (such as, bispecific antibodies). See also, Pierce Catalog and Handbook, 1994-1995 (Pierce Chemical Co., Rockford, IL); Kuby, J., Immunology, 3^{rd} Ed., W.H. Freeman & Co., New York, 1997.

The term “aromatic” generally refers to a cyclic or conjugated group comprising, unless specified otherwise, from 5 to 15 ring atoms having at least a single ring (e.g., phenyl) or multiple condensed rings in which at least one ring is aromatic (e.g., naphthyl, indolyl, or pyrazolopyridinyl); that is, at least one ring, and optionally multiple condensed rings, have a continuous, delocalized π-electron system. Typically, the number of out of plane π-electrons corresponds to the Hiickel rule (4n+2). The point of attachment to the parent structure typically is through an aromatic portion of the condensed ring system. For example,

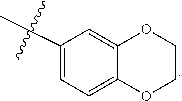 [SMILES: *c1ccc2c(c1)OCCO2]

However, in certain examples, context or express disclosure may indicate that the point of attachment is through a non-aromatic portion of the condensed ring system. For example,

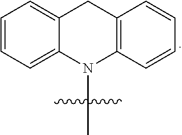 [SMILES: *N1c2ccccc2Cc2ccccc21]

An aromatic group may comprise only carbon atoms in the ring, such as in an aryl group, or it may comprise one or more ring carbon atoms and one or more ring heteroatoms comprising a lone pair of electrons (e.g. S, O, N, P, or Si), such as in a heteroaryl group. Aromatic groups may be substituted with one or more groups other than hydrogen, such as alkyl, heteroalkyl, haloalkyl, aliphatic, heteroaliphatic, haloaliphatic, aryl (such as optionally substituted phenyl or benzyl), heteroaryl, haloaryl, alkylsulfano, or other functionality , or an organic functional group.

The term “aryl” generally refers to an aromatic carbocyclic group comprising at least five carbon atoms to 15 carbon atoms (C₅-C₁₅), such as five to ten carbon atoms (C₅-C₁₀), having a single ring or multiple condensed rings, which condensed rings can or may not be aromatic provided that the point of attachment to a remaining position of the compounds disclosed herein is through an atom of the aromatic carbocyclic group. Aryl groups may be substituted with one or more groups other than hydrogen, such as alkyl, heteroalkyl, haloalkyl, aliphatic, heteroaliphatic, haloaliphatic, aryl (such as optionally substituted phenyl or benzyl), heteroaryl, haloaryl, alkylsulfano, or other functionality.

The term “biological sample” generally refers to hematological, cytological and histological specimens, such as cells, cell cultures, hybridomas, single-celled organisms (e.g. yeast and bacteria), 3D cell cultures (e.g. spheroids and organoids), tissues, whole organisms (e.g. flies or worms), cell-free extracts, or a fluid sample comprising any biological matter (e.g., blood, serum, plasma, sputum, urine, cerebrospinal fluid). Biological samples can be from a plant or animal (e.g., human, mouse, fly, worm, fish, frog, fungi, and the like). A sample can refer to a sample that has been processed by filtration and/or centrifugation and can include supernatants of cell cultures and homogenized tissue or broken up cells.

The term “carbamate” generally refers to —OC(O)NRR′, wherein R and R′ independently are hydrogen, alkyl, heteroalkyl, haloalkyl, aliphatic, heteroaliphatic, haloaliphatic, aryl (such as optionally substituted phenyl or benzyl), heteroaryl, haloaryl, alkylsulfano, or other functionality.

The term “carbonate” generally refers to a functional group with the formula —OCO₂R where R is hydrogen, alkyl, heteroalkyl, haloalkyl, aliphatic, heteroaliphatic, haloaliphatic, aryl (such as optionally substituted phenyl or benzyl), heteroaryl, haloaryl, alkylsulfano, or other functionality.

The term “carboxamide” generally refers to the —N(R)acyl, or —C(O)amino, where R is hydrogen, alkyl, heteroalkyl, haloalkyl, aliphatic, heteroaliphatic, haloaliphatic, aryl (such as optionally substituted phenyl or benzyl), heteroaryl, haloaryl, alkylsulfano, or other functionality.

The term “carboxyl” generally refers to —C(O)OH.

The term “carboxylic acid” generally refers to an organic compound having a formula RCOOH where R is hydrogen, alkyl, heteroalkyl, haloalkyl, aliphatic, heteroaliphatic, haloaliphatic, aryl (such as optionally substituted phenyl or benzyl), heteroaryl, haloaryl, alkylsulfano, or other functionality.

The term “cyano” generally refers to —CN.

The term “disulfide” generally refers to —SSR^{a}, wherein R^{a} is selected from hydrogen, alkyl, heteroalkyl, haloalkyl, aliphatic, heteroaliphatic, haloaliphatic, aryl (such as optionally substituted phenyl or benzyl), heteroaryl, haloaryl, alkylsulfano, or other functionality.

The term “epoxide” generally refers to a cyclic ether with a 3-membered ring having a general formula,

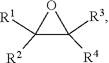

where R¹-R⁴ independently are hydrogen, alkyl, heteroalkyl, haloalkyl, aliphatic, heteroaliphatic, haloaliphatic, aryl (such as optionally substituted phenyl or benzyl), heteroaryl, haloaryl, alkylsulfano, or other functionality.

The term “ester” generally refers to a chemical compound having a formula

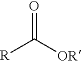

where R and R′ are independently alkyl, heteroalkyl, haloalkyl, aliphatic, heteroaliphatic, haloaliphatic, aryl (such as optionally substituted phenyl or benzyl), heteroaryl, haloaryl, alkylsulfano, or other functionality.

The term “ether” generally refers to a class of organic compounds containing an ether group, that is an oxygen atom connected to two aliphatic and/or aryl groups and having a general formula R—O—R′, where R and R′ are independently alkyl, heteroalkyl, haloalkyl, aliphatic, heteroaliphatic, haloaliphatic, aryl (such as optionally substituted phenyl or benzyl), heteroaryl, haloaryl, alkylsulfano, or other functionality.

The term “equilibration buffer” generally refers to a buffer that is used to infuse a matrix according to the present disclosure to facilitate processing a sample and to promote the affinity of a molecule of interest to the support.

The term “fluorophore” generally refers to a functional group or portion of a compound that causes the compound (or a sample or composition comprising the compound), to fluoresce. In some embodiments, the fluorophore can fluoresce when the compound (or a sample or composition comprising the compound) is exposed to an excitation source or after being cleaved from a compound to which the fluorophore is conjugated.

The term “functional group” generally refers to a specific group of atoms within a molecule that is responsible for the characteristic chemical reactions of the molecule. Exemplary functional groups include, without limitation, alkyl, alkenyl, alkynyl, aryl, halo (fluoro, chloro, bromo, iodo), epoxide, hydroxyl, carbonyl (ketone), aldehyde, carbonate ester, carboxylate, carboxyl, ether, ester, peroxy, hyrdoperoxy, carboxamide, amino (primary, secondary, tertiary), ammonium, imide, azide, cyanate, isocyanate, thiocyanate, nitrate, nitrite, nitrile, nitroalkyl, nitroso, pyridyl, phosphate, sulfonyl, sulfide, thiol (sulfhydryl), disulfide.

The term “halo” generally refers to fluoro, chloro, bromo, or iodo.

The term “heteroaryl” generally refers to an aryl group comprising at least one heteroatom, which can be selected from, but not limited to, oxygen, nitrogen, sulfur, silicon, boron, selenium, phosphorous, and oxidized forms thereof within the ring. Heteroaryl groups can comprise a single ring or multiple condensed rings, wherein the condensed rings may or may not be aromatic and/or contain a heteroatom, provided that the point of attachment is through an atom of the aromatic heteroaryl group. Heteroaryl groups may be substituted with one or more groups other than hydrogen, such as alkyl, heteroalkyl, haloalkyl, aliphatic, heteroaliphatic, haloaliphatic, aryl (such as optionally substituted phenyl or benzyl), heteroaryl, haloaryl, alkylsulfano, or other functionality. In some embodiments, a fluorophore can also be described herein as a heteroaryl group.

The term “hydroxyl” generally refers to the group —OH.

The term “imine” generally refers to an organic compound containing a —C═NR group, where R is hydrogen, alkyl, heteroalkyl, haloalkyl, aliphatic, heteroaliphatic, haloaliphatic, aryl (such as optionally substituted phenyl or benzyl), heteroaryl, haloaryl, alkylsulfano, or other functionality.

The term “Immunoglobin G (IgG)” generally refers to one of the primary classes of immunoglobins having heavy chains known as gamma-chains.

The term “isoelectric point (pI)” generally refers to the pH at which a molecule carries no net electric charge. Polymeric molecules, such as proteins comprised of amino acids, can be positive, neutral, negative, or polar in nature, giving the polymeric material an overall charge. A molecule with a low pI value carries a net negative charge at neutral pH, and a molecule with a high pI value carries a net positive charge at a neutral pH.

The term “molecular weight cut-off” or “MWCO” generally refers to the lowest molecular weight of sample components that are excluded by the matrix, such as the smaller sample components. For example, one or more small molecules, such as, but not limited to BSA, are excluded by the matrix , whereas the larger sample components, such as one or more large molecules, elute faster, and are recovered.

The term “multimodal” generally refers to the ability of a material or compound, such as a resin or matrix, to provide plural different types of interactions between the resin or matrix and a desired molecule to contribute to the separation of a first desired molecule from a second molecule, such as by retention of the first and/or second molecule to the resin or matrix.

The term “negatively charged small molecule” generally refers to a molecule with a low isoelectric point (pI), which carries a net negative charge at a neutral pH.

The term “phosphate” generally refers to —O—P(O)(OR^{a})₂, wherein each R^{a} independently is hydrogen, alkyl, heteroalkyl, haloalkyl, aliphatic, heteroaliphatic, haloaliphatic, aryl (such as optionally substituted phenyl or benzyl), heteroaryl, haloaryl, alkylsulfano, or other organic functional group.

The term “positively charged large molecule” generally refers to a molecule with a high isoelectric point (pI), which carries a net positive charge at a neutral pH.

The term “sample” generally refers to any fluid or solution comprising at least two molecules that need to be separated, wherein a first of the at least two molecules is a small molecule, as defined herein, and a second of the at least two molecules is a large molecule, as defined herein. In some embodiments, the sample may comprise a small molecule, such as but not limited to a stabilizing molecule. The stabilizing molecule can be a purified protein isolated from natural or recombinant sources. For example, a recombinant albumin, a native albumin, human serum albumin, an albumin-like stabilizer, bovine serum albumin, comparable mammalian serum (such as but not limited to, rabbit serum and mouse serum), ovalbumin, glycerol, and/or gelatin. A sample can also include one or more molecules derived from biological samples.

The terms “separation,” “extraction,” “extracted,” “removal,” “reducing” or “reducing the quantity of,” or “purification” generally refer to removing or isolating a substance, e.g., a small molecule, such as BSA, or a large molecule or a biomolecule, such as IgG, from a mixture comprising the small molecule and/or large molecule. An extracted substance or a sample from which a substance has been extracted has significantly decreased quantities of components that were present in the sample prior to separation, and the extracted substance can be substantially reduced, substantially removed, substantially concentrated, substantially pure, or pure (devoid of any contaminants), compared to prior to being extracted.

The present disclosure uses the terms “small molecule” and “large molecule” which refer to species to be separated from one another. “Small molecule” or “smaller molecule” generally refers to any molecule having a molecular weight of less than 100 kDa, and a “large molecule” is a molecule having a molecular weight of greater than or equal to 100 kDa. The small molecule (such as but not limited to a biomolecule) may be being used to treat, derivatize, conjugate, cross-link, label, tag, chemically or biologically modify the large molecule for further analysis. The small molecule can be a stabilizing molecule, such as but not limited to, a purified protein isolated from natural or recombinant sources. For example, a recombinant albumin, a native albumin, human serum albumin, an albumin-like stabilizer, bovine serum albumin, comparable mammalian serum, ovalbumin and/or gelatin. Derivatization includes labeling molecules with labels such as dyes, affinity tags, radioactive labels, mass tags, metals, and the like. Derivatization also includes chemically modifying molecules by reduction, oxidization, methylation, biologically or biochemically modifying biomolecules, etc. Derivatives of biomolecules include, again without limitation: tagged proteins or nucleic acids; labeled biomolecules that are labeled with a variety of labels such as but not limited to dyes, fluorescent dyes, radioactive labels, affinity labels, mass-tags, metals, etc.; conjugated biomolecules, including conjugated antibodies; biomolecules conjugated to nanoparticles; metals, such as gold conjugated to nanoparticles; dyes or labels, such as biotin conjugated to toxins; chemical derivatives of biomolecules, such as but not limited to, reduced proteins, oxidized proteins, methylated nucleic acids, and proteins with sulfhydryl modified proteins. Large molecules and/or biomolecules include, for example and without limitation, proteins, glycoproteins, and antibodies. In one example, the small molecule is BSA having a molecular weight of 67,000 Da. In one example, the large molecule is IgG having a molecular weight of 150 kDa. In certain disclosed examples, a sample comprises a mixture of BSA and IgG.

The term “size exclusion support” generally refers to an inert porous solid that has a porosity which determines the size of a molecule that may be included or excluded from entering the pores. In some disclosed embodiments, the pores of a porous size exclusion support have a molecular size cut-off (MWCO) of 40 kDa or greater than 40 kDa.

The term “sulfonamide” generally refers to the group —SO₂amiI or —N(R)sulfonyl, where R is hydrogen, alkyl, heteroalkyl, haloalkyl, aliphatic, heteroaliphatic, haloaliphatic, aryl (such as optionally substituted phenyl or benzyl), heteroaryl, haloaryl, alkylsulfano, or other functionality.

The term “sulfonate” generally refers to —SO₃⁻, wherein the negative charge of the sulfonate group may be balanced with a positive counterion, such as an M⁺ counter ion, wherein M⁺ may be an alkali ion, such as K⁺, Na⁺, Li⁺; an ammonium ion, such as ⁺N(R^{b})₄, where R^{b} is hydrogen, alkyl, heteroalkyl, haloalkyl, aliphatic, heteroaliphatic, haloaliphatic, aryl (such as optionally substituted phenyl or benzyl), heteroaryl, haloaryl, alkylsulfano, or other functionality; or an alkaline earth ion, such as [Ca²⁺]_{0.5}, [Mg²⁺]_{0.5}, or [Ba²⁺]_{0 0.5}.

The term “sulfonyl” generally refers a functional group with the general formula:

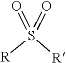

where R represents the rest of the molecule to which the sulfonyl group is bound and R′ is selected from hydrogen, alkyl, heteroalkyl, haloalkyl, aliphatic, heteroaliphatic, haloaliphatic, aryl (such as optionally substituted phenyl or benzyl), heteroaryl, haloaryl, alkylsulfano, or other functionality.

The term “thioester” generally refers to a functional group with the general formula:

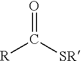

where R represents the rest of the molecule to which the thioester group is bound and R′ is selected from alkyl, heteroalkyl, haloalkyl, aliphatic, heteroaliphatic, haloaliphatic, aryl (such as optionally substituted phenyl or benzyl), heteroaryl, haloaryl, alkylsulfano, or other functionality.

The term “thioether” generally refers to a functional group with the general formula: R—S—R′ where R and R′ independently are selected from alkyl, heteroalkyl, haloalkyl, aliphatic, heteroaliphatic, haloaliphatic, aryl (such as optionally substituted phenyl or benzyl), heteroaryl, haloaryl, alkylsulfano, or other functionality. A thioether is similar to an ether, except that a thioether contains a sulfur atom in place of the oxygen atom of an ether.

All literature and similar materials cited in this application including, but not limited to, patents, patent applications, articles, books, treatises, and internet web pages, regardless of the format of such literature and similar materials, are expressly incorporated by reference in their entirety for any purpose. In the event that one or more of the incorporated literature and similar materials defines or uses a term in such a way that it contradicts that term's definition in this application, the definitions provided by this specification control. While the present teachings are described in conjunction with various embodiments, it is not intended that the present teachings be limited to such embodiments. On the contrary, the present teachings encompass various alternatives, modifications, and equivalents, as will be appreciated by a person of ordinary skill in the art in light of the present teachings.

### III. Matrix

Certain disclosed embodiments of the present disclosure concern a matrix for separating molecules, such as biomolecules, from each other. In some embodiments of the present disclosure, molecules are separated from each other using differences in one or more properties such as the size of the molecules, charge of the molecules, the isoelectric point (pI) of the molecules, and/or a combination of these properties. In certain embodiments, molecules can be separated from each other based on one or more separation matrix properties such charge on the matrix and size exclusion properties of the matrix. The matrix may comprise a porous size exclusion support, at least one cationic moiety, and may further be equilibrated with an equilibration buffer. Each of these components is described in more detail below.

In some embodiments, a sample solution comprising at least one small molecule and at least one large molecule is applied to the matrix, wherein the large molecule elutes faster than the small molecule that is trapped by the matrix. Presently disclosed matrixes provide unexpectedly rapid, economical, and efficient separation of small molecules from large molecules.

One embodiment of the present disclosure describes matrixes for separating, extracting, removing, and/or reducing the quantity of one or more small molecules from one or more large molecules . In some embodiments, the small molecule can be, but is not limited to, a protein, globular protein, sphero protein, serum albumin protein, polypeptide, and/or the like.

In certain disclosed embodiments, the one or more small molecules can be separated from one or more larger molecules using one or more properties, including, but not limited to, the isoelectric point of the molecules. In some embodiments, the one or more small molecules can be one or more negatively charged small molecules. In particular disclosed embodiments, the one or more negatively charged small molecules can have an isoelectric point (pI) value in the range of 4.5-5.5. In some embodiments, the one or more negatively charged small molecules may have a pI value of 4.5, 4.6, 4.7 4.8, 4.9, 5.0, 5.1, 5.2, 5.3, 5.4, and/or 5.5. In one exemplary embodiment, the one or more negatively charged small molecule has a pI value of 4.9.

In some embodiments, the matrix separates, extracts, removes, and/or reduces the quantity of one or more small molecules from one or more large molecules based on, but not limited to, the size of the molecules. In some aspects of the present disclosure, the one or more small molecule can have a molecular weight in the range of less than 100 kDa, less than 80 kDa, and/or less than 70 kDa. In some embodiments, the one or more small molecules may have a molecular weight of from 50 kDa to 80 kDa, such as 50 kDa, 51 kDa, 52 kDa, 53 kDa, 54 kDa, 55 kDa, 56 kDa, 57 kD, 58 kDa, 59 kDa, 60 kDa, 61 kDa, 62 kDa, 63 kDa, 64 kDa, 65 kDa, 66 kDa, 65 kDa, 66 kDa, 67 kDa, 68 kDa, 69 kDa, 70 kDa, 71 kDa, 72 kDa, 73 kDa, 74 kDa, 75 kDa, 76 kDa, 77 kDa, 78 kDa, 79 kDa, and/or 80 kDa. In some exemplary embodiments, the one or more small molecule is BSA with a molecular weight of 67,000 Da (67 kDa).

One embodiment of the present disclosure describes matrixes for separating, extracting, removing, and/or reducing the quantity of one or more small molecules from one or more large molecules. In some of the embodiments, the one or more large molecules can be, but is not limited to, a glycoprotein, phosphoprotein, antibody, or immunoglobulin.

In particular disclosed embodiments, the one or more small molecules can be separated from one or more larger molecules using one or more properties including, but not limited to, the isoelectric point of the molecules. In some embodiments, the one or more large molecules can be one or more positively charged large molecules. In particular disclosed embodiments, the one or more positively charged large molecules may have an isoelectric point (pI) value in the range of 8.0-11.5. In some embodiments, the one or more positively charged large molecules may have a pI value of 8.0, 8.1, 8.2, 8.3, 8.4, 8.5, 8.6, 8.7 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, and/or 11.5. In one exemplary embodiment, the one or more positively charged large molecules has a pI value of 11.0.

In some embodiments, the matrix separates, extracts, removes, and/or reduces the quantity of one or more small molecules from one or more large molecules based on, but not limited to, the size of the molecules. In some embodiments, the one or more large molecules may have a molecular weight greater than or equal to 100 kDa, and in certain embodiments a molecular weight greater than or equal to 150 kDa. For example, 100 kDa, 101 kDa, 102 kDa, 103 kDa, 104 kDa, 105 kDa, 106 kDa, 107 kDa, 108 kDa, 109 kDa, 110 kDa, 110 kDa, 111 kDa, 112 kDa, 113 kDa, 114 kDa, 115 kDa, 116 kDa, 117 kDa, 118 kDa, 119 kDa, 120 kDa, 121 kDa, 122 kDa, 123 kDa, 124 kDa, 125 kDa, 126 kDa, 127 kDa, 128 kDa, 129 kDa, 130 kDa, 131 kDa, 132 kDa, 133 kDa, 134 kDa, 135 kDa, 136 kDa, 137 kDa, 138 kDa, 139 kDa, 140 kDa, 141 kDa, 142 kDa, 143 kDa, 144 kDa, 145 kDa, 146 kDa, 147 kDa, 148 kDa, 149 kDa, 150 kDa, 151 kDa, 152 kDa, 153 kDa, 154 kDa, 155 kDa, 156 kDa, 157 kDa, 158 kDa, 159 kDa, 160 kDa, 161 kDa, 162 kDa, 163 kDa, 164 kDa, 165 kDa, 166 kDa, 167 kDa, 168 kDa, 169 kDa, 170 kDa, 180 kDa, 220 kDa, 250 kDa, 300 kDa, 350 kDa, 450 kDa, 550 kDa, 750 kDa, 900 kDa. In one exemplary embodiment, the large molecule is IgG having a molecular weight of 150,000 Da (150 kDa).

### A. Size Exclusion Support

Matrixes according to the present disclosure may comprise a porous size exclusion support. A porous size exclusion support may comprise spherical beads made of a gel or a gel-like material having pores. The pore size range of a porous size exclusion support determines the size of a molecule that may be included or excluded from entering the size exclusion support. Without being bound by a single theory, it currently is believed that when a sample solution is passed through a size exclusion support, at least one small molecule in the sample enters the pores of the size exclusion support and is forced to follow a circuitous path before later exiting the size exclusion support. On the other hand, larger molecules take a relatively direct path through the size exclusion support. Therefore, without being bound by a particular theory of operation, it is currently believed that the difference in flow rates between the small molecules and the large molecules allows for the separation of the faster-flowing large molecules from the slower-flowing small molecules as a sample travels through the size exclusion support.

Some exemplary size exclusion supports are made of agarose, polyacrylamide, cellulosic materials, and/or derivatives thereof. In some embodiments, a porous size exclusion support may comprise an agarose support, a polyacrylamide support, a cellulosic material support, or derivatives thereof. In some example embodiments, a porous size exclusion support comprises HEC.

In some embodiments, the porous size exclusion support is produced by using sufficient HEC to produce a resin with a MWCO greater than or equal to 40 kDa.

In particular disclosed embodiments, the porous size exclusion support is produced from a range of 50 grams (g) to 250 grams (g) HEC, such as, but not limited to 50 g HEC to 150 HEC g. In some embodiments, 50 g, 51 g, 52 g, 53 g, 54 g, 55 g, 56 g, 57 g, 58 g, 59 g, 60 g, 61 g, 62 g, 63 g, 64 g, 65 g, 66 g, 67 g, 68 g, 69 g, 70 g, 71 g, 72 g, 73 g, 74 g, 75 g, 76 g, 77 g, 78 g, 79 g, 80 g, 81 g, 82 g, 83 g, 84 g, 85 g, 86 g, 87 g, 88 g, 89 g, 90 g, 91 g, 92 g, 93 g, 94 g, 95 g, 96 g, 97 g, 98 g, 99 g, 100 g, 101 g, 102 g, 103 g, 104 g, 105 g, 106 g, 107 g, 108 g, 109 g, 110 g, 111 g,112 g, 113 g, 114 g, 115 g, 116 g, 117 g, 118 g, 119 g, 120 g, 121 g, 122 g, 123 g, 124 g, 125 g, 126 g, 127 g, 128 g, 129 g, 130 g, 131 g, 132 g, 133 g, 134 g, 135 g, 136 g, 137 g, 138 g, 139 g, 140 g, 141 g, 142 g, 143 g, 144 g, 145 g, 146 g, 147 g, 148 g, 149 g 150 g 160 g, 170 g, 180 g, 190 g, 200 g, 201 g, 202 g, 203 g, 204 g, 205 g, 206 g, 207 g, 208 g, 209 g, 210 g, 211 g, 212 g, 213 g, 214 g, 215 g, 216 g, 217 g, 218 g, 219 g, 220 g, 221 g, 222 g, 223 g, 224 g, 225 g, 226 g, 227 g, 228 g, 229 g, 230 g, 240 g, 250 g of HEC can be used to produce the porous size exclusion support. In one exemplary embodiment 80 g of HEC was used to produce the porous size exclusion support. In another exemplary embodiment, 90 g of HEC was used to produce the porous size exclusion support. In another exemplary embodiment, 108 g of HEC was used to produce the porous size exclusion support. In another exemplary embodiment, 129 g of HEC was used to produce the porous size exclusion support. In another exemplary embodiment, 147 g of HEC was used to produce the porous size exclusion support. In another exemplary embodiment, 216 g of HEC was used to produce the porous size exclusion support.

In some aspects of the disclosure, the porous size exclusion support is produced with 60 g HEC to 130 g HEC at a reaction scale of from greater than 0 liters (L) to 10 liters (L), such as, but not limited to, from greater than 0 L to 5 L, to produce the size exclusion support. In some embodiments, 0.5 L to 2 L of resin bed comprising the porous size exclusion support is produced from 60 g HEC to 130 g HEC. In particular disclosed aspect of the present disclosure, 0.5 L to 1.5 L of resin bed comprising the porous size exclusion support is produced from 80 g HEC to 130 g HEC used in a 5 L reaction scale.

In some aspects of the disclosure, the size exclusion support can be crosslinked with a crosslinker such as, but not limited to, an epoxide-containing compound comprising at least one epoxide functional group, such as 1, 2, 3, or 4 epoxide groups. In some embodiments, the epoxide-containing compound may comprise one or more halo functional groups, aliphatic functional groups, heteroaliphatic functional groups, or a combination thereof. In some embodiments, the heteroaliphatic functional group can comprise a polyethylene glycol (or “PEG”) spacer arm. The epoxide-containing compound can have Formula I,

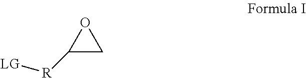

where R is a C₁-C₁₀ alkyl; and LG can be a halo functional group or a glycidol moiety. In one preferred embodiment, the epoxide-containing compound can be epichlorohydrin (Epi), having a structure of

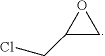

In another preferred embodiment, the epoxide-containing compound can be 1,4-butanediglycidylether.

In some embodiments, the porous size exclusion support can be crosslinked with a range of 250 milliliters (mL) to 700 milliliters (mL) of the crosslinker For example, 250 mL, 251 mL, 252 mL, 253 mL, 254 mL, 255 mL, 256 mL, 257 mL, 258 mL, 259 mL, 260 mL, 261 mL, 262 mL, 263 mL, 264 mL, 265 mL, 266 mL, 267 mL, 268 mL, 269 mL, 270 mL, 271 mL, 272 mL, 273 mL, 274 mL, 275 mL, 276 mL, 277 mL, 278 mL, 279 mL, 280 mL, 281 mL, 282 mL, 283 mL, 284 mL, 285 mL, 286 mL, 287 mL, 288 mL, 289 mL, 290 mL, 291 mL, 292 mL, 293 mL, 294 mL, 295 mL, 296 mL, 297 mL, 928 mL, 299 mL, 300 mL, 301 mL, 302 mL, 303 mL, 304 mL, 305 mL, 306 mL, 307 mL, 308 mL, 309 mL, 310 mL, 311 mL, 312 mL, 313 mL, 314 mL, 315 mL, 316 mL, 317 mL, 318 mL, 319 mL, 320 mL, 321 mL, 322 mL, 323 mL, 324 mL, 325 mL, 326 mL, 327 mL, 328 mL, 329 mL, 330 mL, 331 mL, 332 mL, 333 mL, 334 mL, 335 mL, 336 mL, 337 mL, 338 mL, 339 mL, 340 mL, 341 mL, 342 mL, 343 mL, 344 mL, 345 mL, 346 mL, 347 mL, 348 mL, 349 mL, 355 mL, 356 mL, 357 mL, 358 mL, 359 mL, 360 mL, 361 mL, 362 mL, 363 mL, 364 mL, 365 mL, 366 mL, 367 mL, 368 mL, 369 mL, 370 mL, 371 mL, 372 mL, 373 mL, 374 mL, 375 mL, 376 mL, 377 mL, 378 mL, 379 mL, 380 mL, 381 mL, 382 mL, 383 mL, 384 mL, 385 mL, 386 mL, 387 mL, 388 mL, 389 mL, 390 mL, 391 mL, 392 mL, 393 mL, 394 mL, 395 mL, 396 mL, 397 mL, 398 mL, 399 mL, 400 mL, 401 mL, 402 mL, 403 mL, 404 mL, 405 mL, 406 mL, 407 mL, 408 mL, 409 mL, 410 mL, 411 mL, 412 mL, 413 mL, 414 mL, 415 mL, 416 mL, 417 mL, 418 mL, 419 mL, 420 mL, 421 mL, 422 mL, 423 mL, 424 mL, 425 mL, 426 mL, 427 mL, 428 mL, 429 mL, 430 mL, 440 mL, 450 mL, 460 mL, 470 mL, 480 mL, 500 mL, 520 mL, 540 mL, 560 mL, 580 mL, 600 mL, 620 mL, 640 mL, 660 mL, 680 mL, or 700 mL of the crosslinker can be used, with particular embodiments using Epi as the crosslinker in the above-stated ranges/amounts.

In one exemplary embodiment, 265 mL of Epi was used to crosslink the porous size exclusion support. In another exemplary embodiment, 303 mL of Epi was used to crosslink the porous size exclusion support. In another exemplary embodiment, 375 mL of Epi was used to crosslink the porous size exclusion support. In another exemplary embodiment, 397 mL of Epi was used to crosslink the porous size exclusion support. In another exemplary embodiment, 410 mL of Epi was used to crosslink the porous size exclusion support. In another exemplary embodiment, 441 mL of Epi was used to crosslink the porous size exclusion support. In yet another exemplary embodiment, 662 mL of Epi was used to crosslink the porous size exclusion support.

In some embodiments, the porous size exclusion support can be crosslinked by providing 250 mL Epi to 700 mL Epi at a reaction scale from greater than 0 L to 5 L. In some particular aspects of the present disclosure, greater than 0 L to 2 L of resin bed comprising the porous size exclusion support can be produced from 250 mL Epi to 400 mL Epi 5 L reaction scale.

In some embodiments, the porous size exclusion support has a MWCO greater than or equal to 40 kDa and hence molecules that are excluded have a molecular weight of greater than or equal to 40 kDa. The pore size of the porous size exclusion support may have a MWCO size for excluding from the pores molecules of greater than or equal to 40 kDa to 150 kDa, more typically from 50 kDa to 150 kDa, and even more typically from greater than or equal to 40 kDa to 60 kDa, including a MWCO in between, such as but not limited to, greater than or equal to 40 kDa, 41 kDa, 42 kDa, 43 kDa, 44 kDa, 45 kDa, 46 kDa, 47 kDa, 48 kDa, 49 kDa, 50 kDa, 51 kDa, 52 kDa, 53 kDa, 54 kDa, 55 kDa, 56 kDa, 57 kDa, 58 kDa, 59 kDa, 60 kDa, 61 kDa, 62 kDa, 63 kDa, 64 kDa, 65 kDa, 66 kDa, 67 kDa, 68 kDa, 69 kDa, 70 kDa, 71 kDa, 72 kDa, 73 kDa, 74 kDa, 75 kDa, 76 kDa, 77 kDa, 78 kDa, 79 kDa, 80 kDa, 81 kDa, 82 kDa, 83 kDa, 84 kDa, 85 kDa, 86 kDa, 87 kDa, 88 kDa, 89 kDa, 90 kDa. In one exemplary embodiment, the porous size exclusion support has a MWCO of greater than or equal to 40 kDa. In another exemplary embodiment, the porous size exclusion support has a MWCO of 45 kDa. In another exemplary embodiment, the porous size exclusion support has a MWCO of 50 kDa. In another exemplary embodiment, the porous size exclusion support has a MWCO of 80 kDa. In another exemplary embodiment, the porous size exclusion support has a MWCO of 90 kDa.

### B. Cationic Moiety

Matrixes according to the present disclosure may comprise a porous size exclusion support and a cationic moiety. In some aspects of the present disclosure, small molecules, such as, but not limited to, negatively charged molecules can be separated from large molecules, such as, but not limited to, positively charged molecules based on one or more matrix properties. In some embodiments, the one or more matrix properties can be the charge and/or the size exclusion properties of the matrix.

In some embodiments, the cationic moieties of a matrix of the disclosure are amines, imines, or a combination thereof. In some aspects of the present disclosure, such cationic moieties may comprise an amine-containing polymer; alkylamines, particularly lower alkyl amines (e.g., pentylamines); amine-containing heterocyclic compounds; amine-containing aromatic compounds; or other amine/diamine compounds.

In an exemplary embodiment, prior to association with the porous size exclusion support, the amine-containing polymer is branched polyethylene imine (PEI), having a base structure as shown below, which when exposed to suitable conditions, such as but not limited to, an equilibration buffer, becomes charged to provide the cationic moiety.

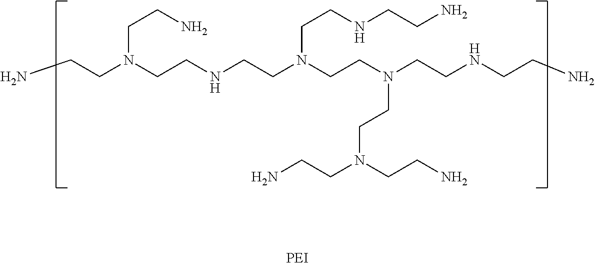

In another exemplary embodiment, prior to association with the porous size exclusion support, the amine-containing polymer is a diamine comprising one or more polyethylene glycol groups, such as 5,8-dimethyl-4,7,10-trioxatridecane-2,12-diamine (also known commercially as Jeffamine) having a base structure as shown below. When exposed to suitable conditions, such as but not limited to, an equilibration buffer, the diamine becomes charged to provide the cationic moiety.

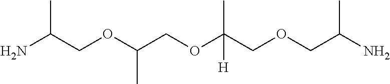 [SMILES: CC(N)COCC(C)OCC(C)OCC(C)N]

### 5,8-Dimethyl-4,7,10-trioxatridecane-2,12-diamine

In another exemplary embodiment, prior to association with the porous size exclusion support, the alkyl diamine is diaminopentane (PDA), having a structure NH₂(CH₂)₅NH₂ (and also shown below), which when exposed to suitable conditions, such as but not limited to, an equilibration buffer, becomes charged to provide the cationic moiety.

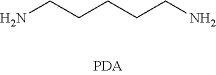

In another exemplary embodiment, prior to association with the porous size exclusion support, the diamine is N,N diethylethylenediamine (DEED), having a structure as shown below, which when exposed to suitable conditions, such as but not limited to, an equilibration buffer, becomes charged to provide the cationic moiety.

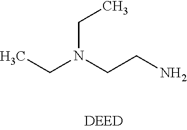

In another aspect of the present disclosure, the amine-containing aromatic compound may comprise at least one aromatic ring having from C₃ to C₁₅ and at least one amine-containing compound. Particular disclosed aspects of the present disclosure, the amine containing-compound can have general Formula II, or an enantiomer, a diastereomer, a tautomer, a salt, a solvate and/or an isotopically substituted derivative thereof

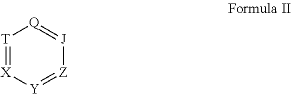

wherein, with reference to Formula II, J, Q, T, X, Y, and Z are the same or different, and each of J, Q, T, X, Y, and Z independently is selected from nitrogen or CR^{c}, wherein R^{c}, for each occurrence, independently is selected from hydrogen, halo, aliphatic, heteroaliphatic, or amino. In some embodiments, each RC can be the same or different. In some embodiments, prior to association with the porous size exclusion support, each of J, Q, T, X, Y, and Z are CR^{c} and at least two R^{c} groups comprise an amino group and the remaining RC groups are hydrogen. In particular embodiments, a compound of Formula II can be selected from, but not limited to, 1,2-diaminobenzene having a structure shown below, 1,3-diaminobenzene having a structure shown below, and/or 1,4-diaminobenzene having a structure shown below. When exposed to suitable conditions, such as but not limited to, an equilibration buffer, each of these compounds becomes charged to provide the cationic moiety.

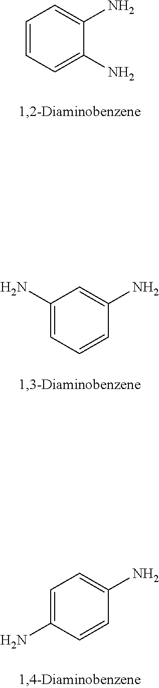 [SMILES: Nc1ccccc1N ; Nc1cccc(N)c1 ; Nc1ccc(N)cc1]

In particular disclosed embodiments, an amine-containing heterocyclic compound can be associated with the size exclusion support comprising HEC. In one example, an epoxy amine compound can be associated with the size exclusion support comprising HEC. In some embodiments, the amine-containing can be an epoxy amine, such as, but not limited to, (S)-N-boc-2,3-epoxypropylamine having a base structure below, which when exposed to suitable conditions, such as but not limited to, an equilibration buffer, becomes charged to provide the cationic moiety.

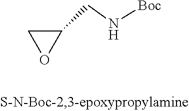 [SMILES: CC(C)(C)OC(=O)NC[C@H]1CO1]

In some embodiments, a matrix of the disclosure comprises at least one cationic moiety that is associated with a porous size exclusion support. In some embodiments, either a porous size exclusion support or a cationic moiety can include a reactive functional group. For example, a functional group on a porous size exclusion support can be used to interact with and associate with one or more cationic moieties to form a matrix.

In some embodiments, a matrix comprises at least one cationic moiety that is immobilized onto at least one size exclusion support. In some embodiments, the cationic moiety may be “immobilized” by being covalently bound to the size exclusion support by formation of a covalent bond. In some embodiments, the covalent bond can be formed using alkylation or by forming an amide or amine bond between a porous size exclusion support and a cationic moiety. In an exemplary embodiment, immobilization according to the disclosure is achieved by first oxidizing hydroxyl groups of HEC to aldehyde groups using any suitable oxidizing agent, with some examples using a periodate (IO⁴⁻ or IO₆⁵⁻). The aldehyde groups generated by oxidation can react with terminal amines on a cationic moiety to covalently bind the cationic moiety to the HEC support, and the resulting intermediate may be reduced by using a reducing agent, such as, but not limited to, sodium cyanoborohydride or picoline borane, to form an amine.

In some embodiments, the epoxy amine can be immobilized onto the size exclusion support. The epoxy amine can associate with and/or immobilize onto the size exclusion support comprising HEC by reacting with a hydroxyl group provided by the HEC. In some aspects of the disclosure, the epoxy amine compound is a protected amine compound, wherein the protected amine compound is deprotected after associating with or immobilizing onto the size exclusion support.

In some embodiments, the cationic moiety associates with the one or more small molecules, such as, but not limited to, a negatively charged small molecule. For example, the negatively charged small molecule can associate with the cationic moiety through interactions and/or bonds, such as, but not limited to, an ionic interaction, a hydrophilic interaction, a hydrophobic interaction, an affinity interaction, hydrogen bonding, Van der Waals forces, and/or covalent bonding.

In particular disclosed embodiments, a functional group on a cationic moiety can react with a functional group on a porous size exclusion support and/or a small molecule, such as but not limited to, a negatively charged small molecule, to be separated or extracted from the at least one large molecule. Functional groups can include, but are not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, and/or any other functional group suitable for associating and/or interacting cationic moieties with the porous size exclusion support and/or small molecule.

In another embodiment, functional groups include at least one reactive group represented by either Rₓ, which represents a reactive functional moiety; or (—L—Rₓ), which represents a reactive functional moiety Rₓ that is attached to either a porous size exclusion support or a moiety by a covalent linkage L. The reactive group functions as the site of association, attachment and/or interaction with a moiety or a small molecule wherein the reactive group chemically reacts with an appropriate reactive or functional group on the porous size exclusion support, the moiety, or the small molecule. In an exemplary embodiment, a reactive group or a functional group can be an acrylamide, an activated ester of a carboxylic acid, an acyl halide group, an acyl azide, an acyl nitrile, an aldehyde, an alkyl halide, an anhydride, an aniline, an aryl halide, an azide, an aziridine, a boronate, a thioboronate group, a carboxylic acid, a diazoalkane, a haloacetamide, a halotriazine, a hydrazine, a hydrazide, an imido ester, an isocyanate, an isothiocyanate, a maleimide, a phosphoramidite, a sulfonyl halide, a thiol group, a sulfide group, a disulfide group, an epoxide group, and episulfide group, a thioester group, an alcohol group, an activated alcohol group, a phosphate group, a phosphate ester group, and/or a photoactivatable group.

In another exemplary embodiment, a reactive group or functional group can comprise electrophiles and/or nucleophiles and can, in some embodiments, generate a covalent linkage between them. Exemplary electrophiles and nucleophile functional groups can an aryloxy group or aryloxy substituted one or more times by electron-withdrawing substituents such as nitro, fluoro, chloro, cyano, trifluoromethyl, or combinations thereof, used to form activated aryl esters; or a carboxylic acid activated by a carbodiimide to form an anhydride or mixed anhydride —OCOR^{a} or —OCNR^{a}NHR^{b}, where R^{a} and R^{b}, which may be the same or different, are C₁-C₆ alkyl, CᵣC₆ perfluoroalkyl, or C₁-C₆ alkoxy; or cyclohexyl, 3-dimethylaminopropyl, an acyl halide group, an acyl nitrile, an aldehyde, an alkyl halide, an anhydride, an aryl halide, an aziridine, a diazoalkane, a haloacetamide, a halotriazine, an isocyanate, an isothiocyanate, a maleimide, a phosphoramidite, a sulfonyl halide, a sulfide group, a disulfide group, an epoxide group, and episulfide group, a thioester group, an activated alcohol group, a phosphate group, a phosphate ester group, and a photoactivatable group. Acyl azides can also rearrange to isocyanates.

In some embodiments, the reactive group further comprises a linker, L, in addition to the reactive functional moiety. The linker can be used to covalently attach a reactive functional group. When present, the linker is a single covalent bond or a series of stable bonds. A reactive functional moiety may be directly attached (where the linker is a single bond) through a series of stable bonds, to the solid support, the moiety or to the small molecule. When the linker is a series of stable covalent bonds the linker typically incorporates several nonhydrogen atoms selected from C, N, O, S, Si, B and P. In addition, the covalent linkage can incorporate a platinum atom, such as described in U.S. Pat. No. 5,714,327. When the linker is not a single covalent bond, the linker may be any combination of stable chemical bonds, optionally including, single, double, triple, or aromatic carbon-carbon bonds, as well as carbon-nitrogen bonds, nitrogen-nitrogen bonds, carbon-oxygen bonds, sulfur-sulfur bonds, carbon-sulfur bonds, phosphorus-oxygen bonds, phosphorus-nitrogen bonds, and nitrogen-platinum bonds. In an exemplary embodiment, the linker incorporates less than 15 nonhydrogen atoms and is composed of a combination of ether, thioether, thiourea, amine, ester, carboxamide, sulfonamide, hydrazide, aromatic, and/or heteroaromatic bonds. Typically, the linker is a single covalent bond or a combination of single carbon-carbon bonds and carboxamide, sulfonamide, or thioether bonds. In some embodiments, the following moieties can be found in the linker: ether, thioether, carboxamide, thiourea, sulfonamide, urea, urethane, hydrazine, alkyl, aryl, heteroaryl, alkoxy, cycloalkyl and amine moieties. Examples of L include substituted or unsubstituted polymethylene, arylene, alkylarylene, aryl, or arylthiol.

Any combination of linkers can be used to attach functional or reactive groups. Where the reactive group is a maleimide or haloacetamide, the resulting compound is particularly useful for conjugation to thiol-containing substances. Where the reactive group is a hydrazide, the resulting compound is particularly useful for conjugation to periodate-oxidized carbohydrates and glycoproteins.

In some embodiments, the cationic moiety may comprise an open and/or unreacted amine. For example, a matrix comprising a size exclusion support and a cationic moiety, such as, but not limited to, PDA, may comprise an open/unreacted amine. In particular disclosed embodiments, the open/unreacted amine on PDA can be reacted with a polysaccharide derived molecule having a molecular weight range from 0 to 2,000,000. In one exemplary embodiment, the open and/or unreacted amine on the cationic moiety PDA is reacted with dextran having a molecular weight of 1,000,000.

In some embodiments, the concentration of the cationic moiety that is added to the matrix can be in the range of from 50 mg/mL to 175 mg/mL. In some embodiments the concentration of the cationic moiety is from 50 mg/mL to 100 mg/mL, from 50 mg/mL to 75 mg/mL, from 50 mg/mL to 150 mg/mL, from 100 mg/mL to 155 mg/mL, from 130 mg/mL to 175 mg/mL. In one exemplary embodiment, the concentration of the cationic moiety can be 50 mg/mL. In another exemplary embodiment, the concentration of the cationic moiety can be 75 mg/mL. In another exemplary embodiment, the concentration of the cationic moiety can be 150 mg/mL.

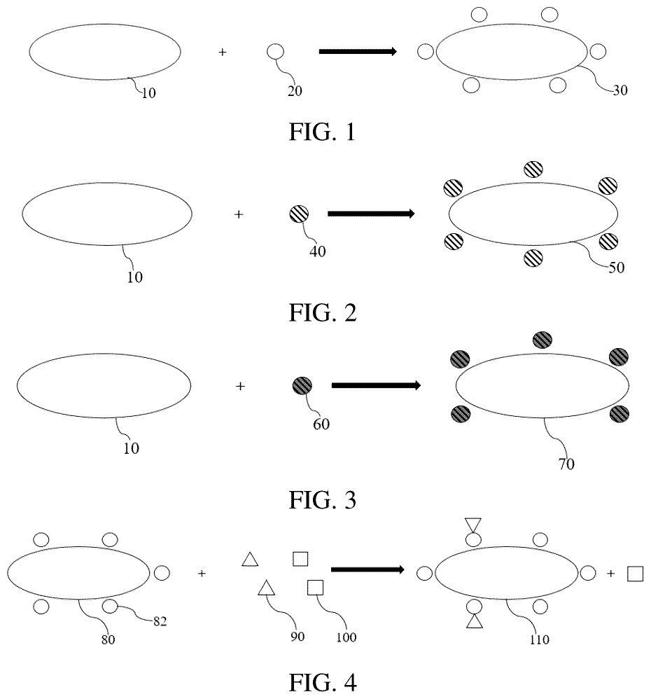

FIG. 1 depicts a non-limiting exemplary embodiment, where a porous size exclusion support 10 is produced with 129 grams of HEC and 265 milliliters of Epi to make 1.5 liters of resin bed, and wherein the HEC is modified with a cationic PDA moiety 20 to form matrix 30. Matrix 30 has a MWCO of 50 kDa, and an overall positive charge.

FIG. 2 depicts another non-limiting exemplary embodiment of a porous size exclusion support 10 that is produced with 129 grams of HEC and 265 milliliters of Epi to make 1.5 liters of resin bed, and wherein the HEC is modified with a branched PEI cationic moiety 40 to form matrix 50. Matrix 50 has a MWCO of 50 kDa, and an overall positive charge.

FIG. 3 depicts another non-limiting exemplary embodiment of a porous size exclusion support 10 is produced with 129 grams of HEC and 265 milliliters of Epi to make 1.5 liters of resin bed, and wherein the HEC is modified with a DEED cationic moiety 60 to form matrix 70. Matrix 70 having a MWCO of 50 kDa, and an overall positive charge.

### C. Equilibration Buffer

In particular aspects of the present disclosure, a buffer can be provided such that it increases the binding capacity of the matrix to smaller sample components while also decreasing the binding capacity of the matrix to larger sample components. For example, and without being bound by a single theory of operation, a buffer increases the binding capacity of the cationic moiety to a small molecule in a sample comprising the small molecule, such as but not limited to, a negatively charged small molecule; and decreases the binding capacity of the cationic moiety to a large molecule, such as, but not limited to a positively charged large molecule. In particular disclosed embodiments, an additional buffer can be used to improve the recovery of larger sample components.

In some embodiments, the buffer is an equilibration buffer. In another embodiment of the present disclosure, the equilibration buffer contains a substantially free of, or is free of, a salt, such as, but not limited to, NaCl. An equilibration buffer comprising a substantially free amount of salt generally refers to greater than 0 mM of salt to 10 mM of salt, preferably from greater than 0 mM of salt to 5 mM of salt. In some aspects, the equilibration buffer can have a pH of 4 to 9 In another embodiment, the equilibration buffer can have a positive charge. In particular disclosed embodiments, the equilibration may comprise a neutral charge.

In some embodiments, the equilibration buffer having a positive charge can include, but is not limited to, carbonate buffers, bicarbonate buffers, phosphate buffers, citric acid/citrate buffers, and combinations thereof. For example, the buffer may comprise a Tris buffer, that is a buffer solution comprising 2-amino-2-(hydroxymethyl)propane-1,3-diol, also referred to as tris(hydroxymethyl)aminomethane; and triethylammonium bicarbonate.

In some aspects of the disclosure, the equilibration buffer can have a pH of 4 to 9, such as from pH of 5 to 8, such as from pH of 5 to 7. In particular aspects of the disclosure, the equilibration buffer has a pH of 5, pH of 7, and/or pH of 8.5. In one exemplary embodiment, the equilibration buffer is sodium acetate having a pH of 5. In another exemplary embodiment, the equilibration buffer is sodium acetate having a pH of 7. In another exemplary embodiment, the equilibration buffer is HEPES having a pH of 5. In another exemplary embodiment, the equilibration buffer is HEPES having a pH of 7. In another exemplary aspect, the equilibration buffer is borate having a pH of 5. In yet another exemplary embodiment, the equilibration buffer is borate having a pH of 8.5.

In some embodiments, the equilibration buffer is a charged buffer with no salt (e.g., no sodium chloride).

In some embodiments, the equilibration buffer is used at a concentration from 1 mM to 100 mM, such as from 10 mM to 100 mM, from 20 mM to 100 mM, from 40 mM to 100 mM, or from 50 to 100 mM. In an exemplary embodiment, the equilibration buffer concentration is 50 mM.

FIG. 4 illustrates a process for separating a small molecule 90 from a sample comprising a large molecule 100 using a matrix 80 having a cationic moiety 82 associated therewith. Small molecule 90 associates with the cationic moiety 82 to form composition 110. A positively charged buffer can be used to facilitate the separation of a small molecule (e.g., a negatively charged molecule, such as BSA), from a large molecule (e.g., a positively charged molecule, such as IgG).

### IV. System

The present disclosure also concerns embodiments of a system comprising one or more disclosed matrixes and further comprising a container. Such systems provide one or more advantages, including, but not limited to: economical feasibility; simplicity and ease of use; providing faster results relative to conventional products in the art; adaptability as a single use disposable unit; adaptability for high throughput sample preparation in multi-well container formats; and adaptability for automated and robotic sample preparation systems. Reducing the concentration of small molecules in a sample using the systems provided here provides quick recovery of biomolecules, as well as superior purity of biomolecules and their derivatives that can be used for downstream applications.

In some embodiments, the present disclosure provides a system for removing one or more small molecules from a sample using differences in one or more properties, such as, but not limited to, the size of the molecules, charge of the molecule, isoelectric point (pI) of the molecules, and/or any combination of these properties. The system can comprise: (i) a container having at least one size exclusion support and at least one cationic moiety that can associate with the one or more small molecules; and (ii) a receptacle located to receive flow from the container. Additionally, molecules can be separated from each other based on one or more separation matrix properties, such as the charge and size exclusion properties of the size exclusion support associated with the at least one cationic moiety. In some disclosed system embodiments, the receptacle is attached to the column. In some embodiments, the receptacle is detachable from the column. The contents of a receptacle can be used or removed by a user as a desired. In some embodiments, the receptacle collects sample with substantially reduced small molecules. In some embodiments, the receptacle collects sample with no small molecules.

The system may be operably configured to operate by gravity flow. Alternatively, an external, affirmatively-applied pressure or force can be applied to facilitate flow, such as a centrifugal force, a positive pressure, a negative pressure, vacuum and combinations thereof. Structures that allow applications of the above-mentioned pressures or forces include, without limitation: a syringe that can be drawn to cause a positive pressure; a vacuum frit for generating negative pressure; and/or tubes or containers adaptable to commercially available centrifuges or rotatory devices. In some embodiments, the system can be configured for use with or in a centrifuge tube or any other comparable rotary instrument.

In some disclosed system embodiments, the container is a columnar container, a tube, a multi-well tube, a multi-well plate or a multi-well filter plate. Exemplary containers include, but are not limited to, a test tube, a spin column, a multi-well plate, a multi-well filter plate, a micro-well plate, or a micro-well filter plate.

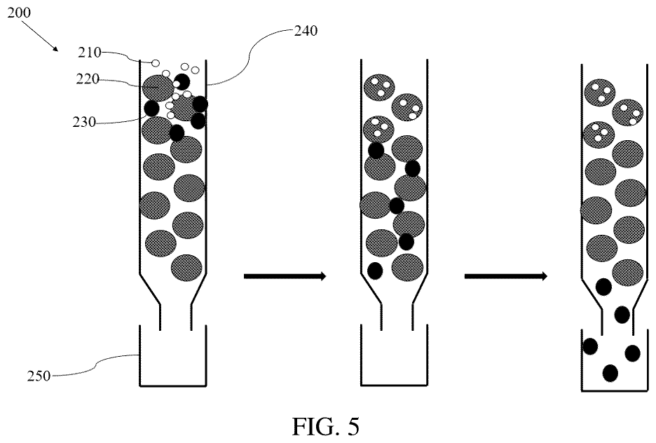

FIG. 5 depicts an exemplary separation system 200 comprising a container 240 and a sample flowing through the container. The sample comprises a small molecule 210 and a large molecule 230. Container 240 houses matrix 220 that is configured to separate the small molecule 210 from the large molecule 230 using differences in one or more properties such as but not limited to, the size of the molecules, the charge of the molecules, the isoelectric point (pI) of the molecules, and/or combination of these properties. System 200 also includes a receptacle 250 for recovering the large molecule 230.

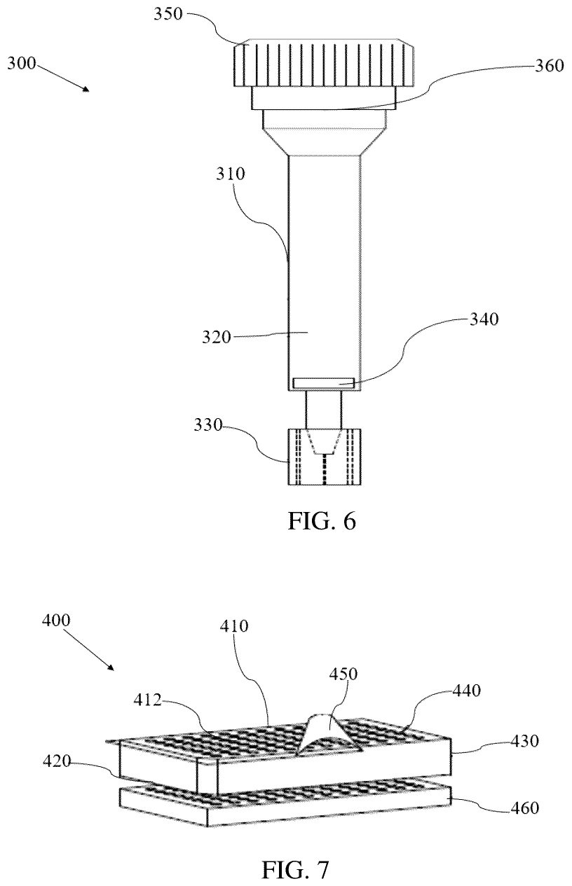

FIG. 6 depicts an exemplary separation system 300 according to one embodiment of the present disclosure. System 300 comprises: a container 310 (such as a columnar tube, a test-tube or a spin column); a matrix 320 housed by the container 310; a receptacle 330 located below the container 310 that is adaptable or configured to receive fluid flowing through the container 310 through its bottom end 340. In some embodiments, container 310 may also comprise one or more frits (not depicted). In some embodiments, system 300 can include an optional lid 350 that can be used to secure container 310 at a top end 360. In some embodiments, receptacle 330 can be detached from container 310 so that a user can collect the flow-through. In some embodiments, receptacle 330 has a twist-off tab configuration for removal. In other embodiments a receptacle 330 can be threadedly coupled to container 310, or attached using a complementary fit that can be pulled apart, and the like.

FIG. 7 is a perspective view of an exemplary system 400 according to one embodiment of the present disclosure comprising a multi-well container 410. Multi-well container 410 comprises a wall that defines multiple wells 412. The wall may comprise an end 420, a side 430, and a top 440 that defines multiple wells. Multi-well container 410 houses a matrix comprising at least one size exclusion support and at least one cationic moiety according to the present disclosure that can associate with the one or more small molecules using the properties, such as, but not limited to, the size, the charge, the isoelectric point (pI), and/or any combination of these properties. As illustrated by FIG. 7, the multi-well container may comprise an optional container lid 450. Lid 450 can be any suitable removable/detachable structure, such as a foil, a clear wrap, or a tear-off seal. Multi-well container 410 can be a multi-well plate, a multi-well plate filter, a microplate or a microtiter plate comprising a flat plate comprising multiple-wells 412 where each well is used as a small test tube or container. Multi-well plates come in a variety of formats for high-throughput use and may comprise 6, 12, 24, 48, 96, 384, 1536, 3456, 9600 or more wells arranged in a rectangular matrix or array.

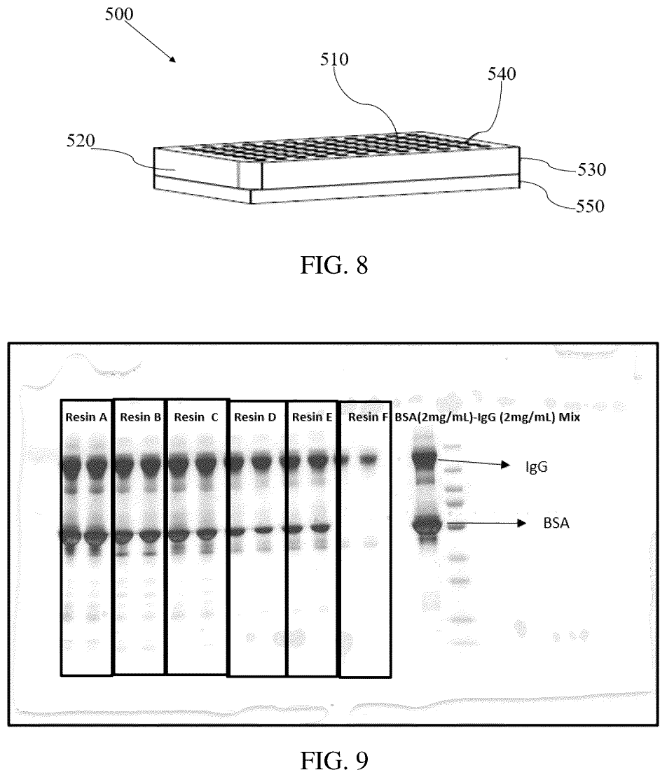

FIG. 8 depicts an exemplary system 500 according to one embodiment comprising a multi-well container 510. System 500 further comprises a receptacle 550 located below the container 510 that is adaptable or configured to receive fluid flowing from the container. Container 510 comprises an end 520, a top 540, and a side 530. In some embodiments, receptacle 550 may comprise a multi-well tray to collect flow-through. Receptacle 550 may be detachable and can be collected by a user. In some embodiments, receptacle 550 is a wash plate or a collection plate.

Systems according to the present disclosure may be fully automated or may be manually operated systems. In some embodiments, a system may be operated in part manually and in-part by automation.

A system can also comprise a computer system comprising a CPU, hardware elements, and/or software elements. Suitable computer systems may be operable to control various components of the system, such as a control robot to retrieve flow-through and analyze flow-through. In some embodiments, a computer system and/or components thereof may reside physically within system 300, 400, or 500, or may reside externally. A computer system used herein may comprise a data analysis and control system, a data transfer system such as a read-write CD ROM Drive or DVD drive, at least one USB port, and/or at least one Ethernet port. In some embodiments, a computer system may include pre-loaded software and/or Application Specific Integrated Circuits (ASICS) to control disclosed systems, such as systems 300, 400, 500, and/or other components the system, including sample processing and analysis, display, and/or exporting the results.

Disclosed system embodiment also may optionally comprise one or more devices or components operable to further process the flow-through. In some embodiments, the flow-through can be eluted molecules, such as but not limited to, eluted larger sample components, such as one or more large molecules.

A system may also comprise additional devices or components, such as but not limited to, a power supply, a display unit, such as a monitor operable to view sample processing and/or to monitor extraction of biomolecules from samples; spectrophotometers; devices to measure nucleic acid extraction; devices to further process extracted biomolecules for further analysis; printers and the like. A system of the disclosure may be configured to fit on a laboratory bench top.

### V. Method of Making

Embodiments of the present disclosure also concern a method for making a matrix or system according to the present disclosure. In some embodiments, the method of making comprises immobilizing a cationic moiety to a porous size exclusion support.

A porous size exclusion support may comprise spherical beads made of a gel or a gel-like material having pores. Some exemplary size exclusion supports comprise agarose, polyacrylamide, cellulosic materials (e.g., hydroxyethyl cellulose), and/or derivatives thereof.

In some embodiments, the porous size exclusion support may be crosslinked with at least one crosslinker. The pore size range of a porous size exclusion support determines the size of a molecule that may be included or excluded from entering the porous size exclusion support. Without being bound by this theory, it currently is believed that when a sample solution is passed through a porous size exclusion support molecules having a molecular weight less than or substantially equal to the MWCO are forced to follow a circuitous path before later exiting the porous size exclusion support. On the other hand, large molecules take a relatively direct path through the porous size exclusion support. Therefore, the difference in flow rates between the small molecules and large molecules allows for separating the faster-flowing large molecules from the slower-flowing small molecules as a sample travel through the size exclusion support.

Disclosed embodiments of the porous size exclusion support may comprise products formed by reacting 50 grams (g) to 250 grams (g) HEC. In some embodiments, disclosed size exclusion supports have may comprise HEC ranging from 80 grams to 130 grams HEC for a 1.5 L resin bed. In one exemplary embodiment 80 g of HEC was used to produce the porous size exclusion support. In another exemplary embodiment, 90 g of HEC was used to produce the porous size exclusion support. In another exemplary embodiment, 108 g of HEC was used to produce the porous size exclusion support. In another exemplary embodiment, 129 g of HEC was used to produce the porous size exclusion support. In another exemplary embodiment, 147 g of HEC was used to produce the porous size exclusion support. In another exemplary embodiment, 216 g of HEC was used to produce the porous size exclusion support.

In some embodiments, the porous size exclusion support is produced by crosslinking HEC with a crosslinker. For example, HEC can be crosslinked with an epoxide, such as but not limited to, epichlorohydrin (Epi). In some embodiment, the porous size exclusion support can be crosslinked with a range of 250 mL to 450 mL Epi. The support can be produced from 250 mL to 450 mL of epichlorohydrin for a 1.5 L resin bed. In one exemplary embodiment, 265 mL of Epi was used to crosslink the porous size exclusion support. In another exemplary embodiment, 303 mL of Epi was used to crosslink the porous size exclusion support. In another exemplary embodiment, 375 mi., of Epi was used to crosslink the porous size exclusion support. In another exemplary embodiment, 397 mL of Epi was used to crosslink the porous size exclusion support. In another exemplary embodiment, 410 mL of Epi was used to crosslink the porous size exclusion support. In another exemplary embodiment, 441 mL of Epi was used to crosslink the porous size exclusion support.

In some embodiments, the cationic moiety is immobilized to the porous exclusion support by formation of a covalent bond. In some embodiments, the covalent bond can be formed by reactions, such as, but not limited to, amidation, alkylation, amination, or other covalent bond-forming reactions. In some such embodiments, a functional group of the size exclusion support (e.g., hydroxyl group) is oxidized, such as by using a periodate, to generate an aldehyde. These generated aldehyde groups can react with terminal amines on a cationic moiety to form an imine intermediate that can be chemically reduced to form an amine.

In some embodiments, 15-35 mg/mL of the oxidation agent, such as a periodate, is added to oxidize the size exclusion support. In some embodiments, 15 mg/mL, 16 mg/mL, 17 mg/mL, 18 mg/mL, 19 mg/mL, 20 mg/mL 21 mg/mL, 22 mg/mL 23 mg/mL, 24 mg/mL 25 mg/mL, 26 mg/mL, 27 mg/mL, 28 mg/mL, 29 mg/mL, 30 mg/mL, 31 mg/mL, 32 mg/mL, 33 mg/mL, 34 mg/mL, 35 mg/mL, of a periodate is added to oxidize the size exclusion support. In some embodiments, the size exclusion support is oxidized using sodium periodate.

In some embodiments, 5-15 mg/mil, of the reducing agent is added. In some embodiments, 5 mg/ mL, 6 mg/mL, 7 mg/mL, 8 mg/mL, 9 mg/mL, 10 mg/mL, 11 mg/mL, mg/mL, 13 mg/mL, 14 mg/mL, 15 mg/mL, of the reducing agent is added. In an exemplary embodiment, the reducing agent is sodium cyanoborohydride. In another exemplary embodiment, the reducing agent is picoline borane.

In some embodiments, the cationic moieties associated with a size support may comprise an amine group. In some embodiments, the cationic moiety comprises an amine-containing polymer, pentylamine groups, diamine, or an imine group. In an exemplary embodiment, the amine-containing polymer is branched PEI. In another exemplary embodiment, the diamine is PDA. In another embodiment, the diamine is DEED.

In some embodiments, the concentration of the cationic moiety that is used to modify the support material can be in the range of 50 mg/mL to 175 mg/mL. In some embodiments, the concentration of the cationic moiety may comprise from 50 mg/mL to 170 mg/mL, from 60 mg/mL to 165 mg/mL, from 65 mg/mL to 160 mg/mL, from 70 mg/mL to 155 mg/mL, from 75 mg/mL to 150 mg/mL, from 80 mg/mL to 145 mg/mL, from 85 mg/mL to 140 mg/mL, from 90 mg/mL to 135 mg/mL, or from 95 mg/mL to 130 mg/mL. In one exemplary embodiment, the concentration of the cationic moiety is 50 mg/mL. In another exemplary embodiment, the concentration of the cationic moiety 75 mg/mL. In yet another exemplary embodiment, the concentration of the cationic moiety can be 150 mg/mL.

In an exemplary embodiment, sodium metaperiodate is dissolved in water and is mixed with the porous size exclusion support matrix bed comprising ITEC to oxidize vicinal diols present in the HEC to aldehyde groups. The mixture is allowed to react for a suitable time period, such as from 2 to 4 hours, at a temperature ranging from 15° C. to 30° C. In an exemplary embodiment, the mixture is allowed to react for at least four hours at room temperature with constant overhead stirring. In some embodiments, during the reductive amination step (i.e., reacting the amine group on the cationic moiety with the aldehyde groups on size exclusion support matrix bed and reducing intermediate imines to amines) the cationic moiety is prepared at a pH from 8.0 to 8.5 and is added to the slurry. A suitable reducing agent, such as sodium cyanoborohydride, is added to the mixture, and the reaction is allowed to proceed for a suitable time period, such as from 8 to 12 hours at 20° C. 30° C. with stirring, and is washed with water and NaCl.

### VI. Method of Using

Certain disclosed embodiments concern a method for separating at least one large molecule from at least one small molecule using differences in one or more properties, such as, but not limited to, size of the molecule, charge of the molecules, the isoelectric point (pI) of the molecules, and/or any combination of these properties. The method may comprise: applying a sample to a porous size exclusion support comprising at least one cationic moiety that can bind to or associate with a small molecule; and subjecting the container to a gravity flow, a centrifugal force, a positive pressure, a negative pressure, a vacuum or a combination thereof. The large molecule in the sample can be excluded by the porous size exclusion support and is collected as flow-through. The one small molecule can interact with the cationic moiety and is thereby separated from the large molecule in a sample using differences in one or more properties such as but not limited to the size of the molecules, the charge of the molecules, the isoelectric point (pI) of the molecules, and/or any combination of these properties.

In particular aspects of the present disclosure, an equilibration buffer disclosed herein can be provided such that it increases the binding capacity of the matrix to smaller sample components while also decreasing the binding capacity of the matrix to larger sample components.

Currently known methods of removing small molecules, such as, but not limited to, BSA from larger molecules, such as, but not limited to, an antibody, in sample requires an initial step, such as pre-salting step to remove salt from the antibody solution. Without being bound by a theory of operation, the salt creates a counterion and would reduce removal of BSA in a subsequent step. The present disclosure allows for the separation of a small molecule, such as, but not limited to, BSA, to be removed from larger molecules, such as, but not limited to, an antibody, in one step. As such, embodiments of the present disclosure do not require a pre-desalting step to achieve a desirable BSA removal and antibody recover, such as, but not limited to, IgG. Accordingly, in some embodiments, separation of a small molecule from the remainder of the sample is carried out in one step.

In some embodiments, flow-through is collected in a receptacle located below the container. In some embodiments, small molecules may constitute a sample impurity or contaminant.

Samples that may be processed by methods of the disclosure may be any type of biological or clinical sample comprising biomolecules or derivatives thereof from which small molecules have to be separated or removed. Some exemplary non-limiting samples include samples having the small molecule BSA and the large molecule IgG.

Methods of the disclosure advantageously reduce the time required for processing a sample, and/or increase the quantity of small molecules removed from the sample.

### VII. Kits

The present disclosure also describes kits for implementing the methods discussed herein and/or kits that contain matrixes and/or kits that contain systems discussed herein.

In some embodiments, the present disclosure describes a kit for separating a large molecule from a small molecule using differences in one or more properties, such as, but not limited to, the size of the molecules, charge of the molecules, the isoelectric point of the molecules, and/or any combination of these properties. The kit may comprise: a system comprising (i) a container housing a size exclusion support comprising a cationic moiety associated with the support, wherein the cationic moiety also can associate with a small molecule; and (ii) a receptacle located below the container, wherein the device is operably configured for gravity flow, or can operate by applying a centrifugal force, a positive pressure, a negative pressure, a vacuum, and combinations thereof.

In some embodiments of a kit of the disclosure the device is a spin column, a multi-well filter plate, or a multi-well plate. A kit can further comprise one or more equilibration buffers packaged in one or more separate containers or included in the first container.

In some embodiments, the equilibration buffer does not comprise a salt (e.g., an ionic salt like NaCl). In other aspects disclosed herein, the equilibration buffer may comprise a low amount of salt. In some aspects, the equilibration buffer can have a pH of 4 to 9 In another embodiment, the equilibration buffer can have a positive charge. In particular disclosed embodiments, the equilibration may comprise a neutral charge.

In some embodiments, the equilibration buffer can include but is not limited to, carbonate buffers, bicarbonate buffers, phosphate buffers, citric acid/citrate buffers. In an exemplary embodiment, the buffer comprises a Tris Buffer. In another exemplary embodiment, the buffer comprises a TEAB buffer.

In some aspects of the present disclosure, the equilibration buffer can have a pH of 4-9, such as from pH of 5 to 8, such as from pH of 5 to 7. In particular aspects, the equilibration buffer has a pH of 5, pH of 7, and/or pH of 8.5. In one exemplary embodiment, the equilibration buffer is sodium acetate having a pH of 5. In another exemplary embodiment, the equilibration buffer is sodium acetate having a pH of 7. In another exemplary embodiment, the equilibration buffer is HEPES having a pH of 5. In another exemplary embodiment, the equilibration buffer is HEPES having a pH of 7. In another exemplary aspect, the equilibration buffer is borate having a pH of 5. In yet another exemplary embodiment, the equilibration buffer is borate having a pH of 8.5.

In some embodiments, the positively charged equilibration buffer is present in a concentration from 1 mM to 100 mM, from 2 mM to 75 mM, from 5 mM to 50 mM, from 10 mM to 25 mM, from 5 to 25 mM. In some embodiments, the positively charged equilibration buffer is present in a concentration from 1 mM to 100 mM, from 2 mM to 75 mM, from 5 mM to 50 mM, from 10 mM to 25 mM, from 5 to 25 mM Tris Buffer. In an exemplary embodiment, the equilibration buffer concentration may be 50 mM.

A kit of the disclosure may also comprise one or more reagents such as one or more wash buffers, elution buffers, filter membranes and/or additional spin columns or multi-well plates.

Reagents and components of kits may be comprised in one or more suitable containers. A container may generally comprise at least one vial, test tube, flask, bottle, syringe or other container, into which a component may be placed, and preferably, suitably aliquoted. Where there is more than one component in a kit they may be packaged together if suitable or the kit will generally contain a second, third or other additional container into which the additional components may be separately placed. However, in some embodiments, certain combinations of components may be packaged together comprised in one container means. A kit can also include a component for containing any reagent containers in close confinement for commercial sale. Such containers may include injection or blow-molded plastic containers into which the desired vials are retained.

In some embodiments, a component of a kit of the disclosure may be pre-filled with one or more of the reagents to process a sample and may be suitably aliquoted into appropriate chambers. A kit or containers thereof may have a seal to keep the internal compartments and any contents therein sterile and spill proof.

Some components of a kit are provided in one and/or more liquid solutions. Liquid solution may be non-aqueous solution, an aqueous solution, and may be a sterile solution. Components of the kit may also be provided as dried powder(s). When reagents and/or components are provided as a dry powder, the powder can be reconstituted by the addition of a suitable solvent. It is envisioned that a suitable solvent may also be provided in another container means. Kits may also comprise a container means for containing a sterile, pharmaceutically acceptable buffer and/or other diluent.

A kit of the disclosure may also include instructions for employing the kit components and may also have instructions for the use of any other reagent not included in the kit. Instructions can include variations that can be implemented.

### VIII. Overview of Several Embodiments

Disclosed herein are embodiments of a matrix, comprising: a porous size exclusion support; and at least one cationic moiety associated with the porous size exclusion support, wherein the matrix can separate one or more molecules in a sample by using a molecular weight of the one more molecules, a charge of the one or more molecules, an isoelectric point (pI) of the one or more molecules, or a combination thereof.

In any or all embodiments, the porous size exclusion support has a molecular weight cut-off of greater than or equal to 40 kDa.

In any or all embodiments, the at least one cationic moiety is covalently bound to the porous size exclusion support.

In any or all embodiments, the at least one cationic moiety comprises an amine, a diamine, a polyamine, or an amine-containing polymer.

In any or all embodiments, the amine-containing polymer is polyethylene imine.

In any or all embodiments, the diamine is diaminopentane, N,N-diethylethylenediamine, or combination thereof.

In any or all embodiments, the porous size exclusion support comprises hydroxyethyl cellulose.

In any or all embodiments, the further comprises epichlorohydrin.

Also disclosed herein are embodiments of a system for separating one or more molecules in a sample using a molecular weight of the one more molecules, a charge of the one or more molecules, an isoelectric point (pI) of the one or more molecules, or a combination thereof, the system comprising a container comprising the matrix of the above embodiments, and a receptacle located to receive flow from the container.

In any or all embodiments, the system configured for gravity flow operation, centrifugal force operation, positive pressure operation, negative pressure operation, vacuum operation, or combinations thereof.

In any or all embodiments, the container is a columnar container, a tube, a multi-well tube, a multi-well plate, or a multi-well filter plate.

Also disclosed herein is a method of making a multimodal resin, comprising: providing a porous size exclusion support comprising hydroxyethyl cellulose having a MWCO of greater than or equal to 40 kDa, the hydroxyethyl cellulose having at least one vicinal diol; oxidizing the least one vicinal diol to an aldehyde; and reacting the aldehyde with an amine group of a cationic moiety via reductive amination.

In any or all embodiments, the porous size exclusion support is produced using an amount of hydroxyethyl cellulose ranging from 50 g to 250 g.

In any or all embodiments, the method further comprising providing a crosslinker to crosslink the porous size exclusion support.

In any or all embodiments, the crosslinker is epichlorohydrin.

In any or all embodiments, the epichlorohydrin is used in an amount ranging from 250 mL to 450 mL.

In any or all embodiments, the cationic moiety is diaminopentane, polyethylene imine, or N,N-diethylethylenediamine.

In any or all embodiments, the cationic moiety has a concentration range from 50 mg/mL to 175 mg/mL.

Also disclosed herein is a method for separating one or more molecules in a sample, comprising: providing a matrix comprising a porous size exclusion support having at least one cationic moiety associated therewith, wherein the matrix can separate one or more molecules using a molecular weight of the one or more molecules, a charge of the one or more molecules, an isoelectric point (pI) of the one or more molecules, or a combination thereof; equilibrating the matrix with an equilibration buffer; and applying the sample to the matrix to separate the one or more molecules by subjecting the matrix to gravity flow, a centrifugal force, a positive pressure, a negative pressure, a vacuum, or a combination thereof, wherein the one or more molecules in the sample is excluded by the matrix and is collected as a flow-through, and wherein one or more molecules associates with the at least one cationic moiety and is thereby separated from the sample in a single step.

In any or all embodiments, the flow-through is collected in a receptacle located below the container.

In any or all embodiments, the equilibration buffer is a positively charged buffer, a neutrally charged buffer, a low-salt buffer, or a salt-free buffer.

In any or all embodiments, the equilibration buffer is sodium acetate having a pH of 5 or a pH of 7, HEPES having a pH of 5 or a pH of 7, or borate having a pH of 5 or a pH of 8.5.

In any or all embodiments, the positively charged buffer is Tris Buffer or triethylammonium bicarbonate.

In any or all embodiments, the first of the two or more molecules comprises: at least one small molecule having a molecular weight range of less than 100 kDa; and the second of the one or more molecules comprises least one large molecule having a molecular weight range of greater than or equal to 100 kDa.

In any or all embodiments, the one or more molecules is bovine serum albumin.

In any or all embodiments, the one or more molecules is an antibody. In any or all embodiments, the antibody is IgG.

In any or all embodiments, the one or more molecules having an isoelectric point value range from 4.5 to 5.5 are separated from one or more molecules having an isoelectric point value from 8.0 to 11.5.

In any or all embodiments, the one or more molecules are more negatively charged molecule from the other having positively charged molecules.

In any or all embodiments, the at least one cationic moiety associates with the one or more molecules by ionic interaction, hydrophilic interactions, hydrophobic interactions, affinity interaction, hydrogen bonding, or van der Waals forces.

In any or all embodiments, the one or more molecules comprises at least one negatively charged small molecule and at least one positively charged large molecule, and the size exclusion support comprises HEC and Epi, and the cationic moiety is diaminopentane; wherein greater than 80% of the at least one negatively charged molecule is separated and greater than 80% of the of the at positively charged large molecule is recovered as flow-through.

In any or all embodiments, the one or more molecules comprises at least one negatively charged small molecule and at least one positively charged large molecule, and size exclusion support comprises HEC and Epi, cationic moiety is diaminopentane, and the cationic moiety is modified with dextran; wherein greater than 80% of the at least one negatively charged molecule is separated and greater than 80% of the of the at positively charged large molecule is recovered as flow-through.

In any or all embodiments, the one or more molecules comprises at least one negatively charged small molecule and at least one positively charged large molecule, and size exclusion support comprises HEC and Epi, and the cationic moiety is N,N-diethylethylenediamine; wherein greater than 50% of the at least one negatively charged molecule is separated and greater than 90% of the of the at positively charged large molecule is recovered as flow-through.

In any or all embodiments, the one or more molecules comprises at least one negatively charged small molecule and at least one positively charged large molecule, and size exclusion support comprises HEC and Epi, and the cationic moiety is diaminopentane; the equilibration buffer is Tris buffer; and wherein greater than 75% of the at least one negatively charged molecule is separated and greater than 80% of the of the at positively charged large molecule is recovered as flow-through.

In any or all embodiments, the negatively charged molecule is bovine serum albumin and the cationic moiety has binding capacity of 1.6 mg/mL for bovine serum albumin when the cationic moiety is diaminopentane, and the equilibration buffer is Tris buffer.

In any or all embodiments, the negatively charged molecule is bovine serum albumin and the cationic moiety has binding capacity of 3.11 mg/mL for bovine serum albumin when the cationic moiety is polyethylene imine, and the equilibration buffer is Tris buffer.

In any or all embodiments, the one or more molecules comprises at least one negatively charged small molecule and at least one positively charged large molecule, and size exclusion support comprises HEC, and the cationic moiety is polyethylene imine; the equilibration buffer is Tris buffer; and wherein greater than 70% of the of the at positively charged large molecule is recovered as flow-through.

Also disclosed herein are embodiments of a kit for separating a positively charged large molecule from one or more negatively charged small molecules in a sample, the kit comprising: a porous size exclusion support having at least one cationic moiety associated therewith, wherein the cationic moiety can associate with and capture the at least one negatively charged small molecule; and instructions for using the porous size exclusion support.

In any or all embodiments, the kit further comprises an equilibration buffer.

In any or all embodiments, the equilibration buffer is Tris buffer or triethylammonium bicarbonate.

In any or all embodiments, the kit further comprises a system comprising: a container housing the porous size exclusion support; and a receptacle positioned to receive flow-through the support.

In any or all embodiments, the system of the kit is configured to operate by gravity flow, a centrifugal force, a positive pressure, a negative pressure, vacuum, and combinations thereof.

In any or all embodiments the container of the kit is a spin column, a multi-well filter plate, or a multi-well plate.

### IX. EXAMPLES

Aspects of the present teachings can be further understood in light of the following examples. Matrixes comprising at least one size exclusion support and at least one cationic moiety for separating and/or extracting one or more small molecules from a sample that can associate with the one or more small molecules using differences in one or more properties, such as but not limited to, the size of the molecules, the charge of the molecules, the isoelectric point of the molecules, and/or any combination of these properties were prepared and tested. In some embodiments, exemplary size exclusion supports were modified with cationic moieties comprising functional groups that can associate with negatively charged small molecules by either ionic interaction, hydrophilic interaction, or any other interaction(s) that can associate with and thereby remove negatively charged small molecules from a sample while allowing at least one large positively charged molecules in the sample to be excluded and collected.

In the following examples, high concentrations of BSA (67,000 Da) were separated from IgG (150,000 Da) from samples comprising a BSA-IgG mixture. Pierce™ Rapid Gold BCA Protein Kit was utilized to measure the respective concentrations of BSA and IgG when the proteins were spun through separately. Quantitative iBright analysis of SDS-Page results provided an accurate estimation of BSA and IgG recovery rates when IgG-BSA mixture was spun through the resin. Moreover, the following examples address the issue of BSA directly competing with IgG during antibody labeling without adversely affecting the original amount of IgG and demonstrate superior performance versus current commercial resins in separating BSA from IgG.

Furthermore, in some of the following examples, BSA removal and IgG purification kits were used. For example, the Abcam BSA Removal Kit (ab173231) commercially sold by Abcam and kits comprising a Melon Gel resin used for binding and removing serum proteins such as the Melon™ Gel IgG Purification Kit commercially sold by Thermo Scientific™ were used in the following examples. Moreover, crosslinked agarose beads bound to Cibacron Blue F3GA dye such as the Affi-Gel® Blue Gel commercially sold by Bio-Rad was used in the following examples.

While these exemplary small molecules and the listed molecular weight ranges were used in the experimental demonstration, a person of ordinary skill in the art will realize that the present embodiments are not limited to either these small molecules or molecular weight ranges and the teachings herein enable a person of ordinary skill in the art to make and use matrixes and systems for removal of a variety of small molecules and molecular weight ranges.

### Example 1

Preparation Chemistry: A porous size exclusion support comprising HEC crosslinked with Epi in the presence of a non-polar phase containing solvent and a surfactant (i.e., nonionic detergent)) was modified to produce the embodiments of Table 1 according to Scheme 1.

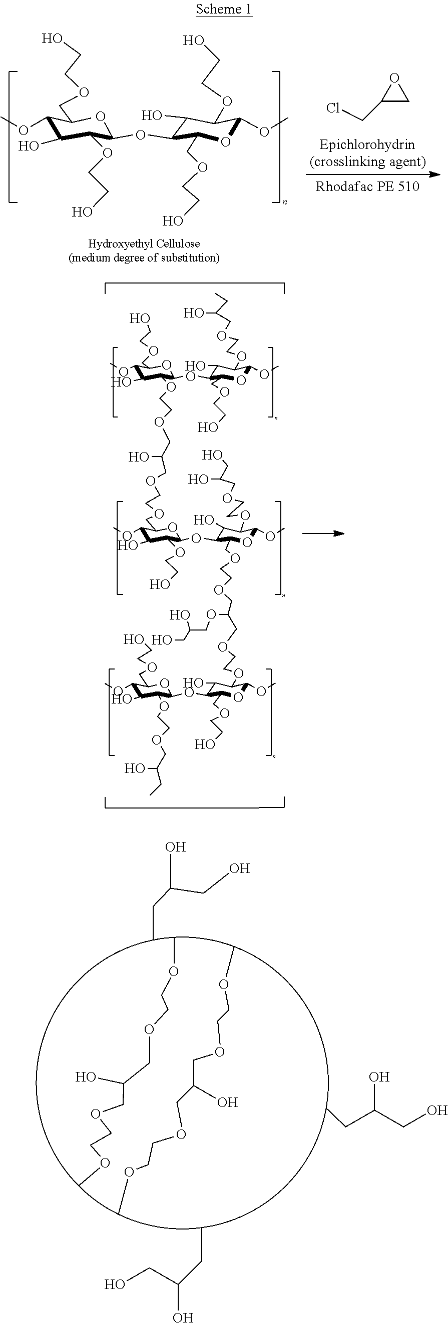

TABLE 1

| | | Amount of HEC | Amount of Epi |
|---|---|---|---|
| | MWCO (Dalton) | (Grams) | (Milliliters) |
| Resin 1 | 2000 | 216 | 662 |
| Resin 2 | 7000 | 216 | 375 |
| Resin 3 | 40,000 | 129 | 397 |
| Resin 4 | 30,000 | 147 | 397 |
| Resin 5 | 45,000 | 129 | 265 |
| Resin 6 | 50,000 | 108 | 410 |
| Resin 7 | 80,0000 | 90 | 441 |
| Resin 8 | 90,0000 | 80 | 303 |

Next, vicinal diols, located on these size exclusion support columns, were oxidized using a periodate to generate aldehyde groups. PDA, branched PEI, or DEED, were prepared in PBS, the pH was adjusted to a range of 8.0-8.5, and reacted with the oxidized columns according to the following procedure. Different Size Exclusion Support's (see Table 1) with different MWCOs were reacted with cationic reagents PDA and DEED to produce the Multimodal Resins of Table 2 according to Scheme 2.

TABLE 2

| Multimodal Resin | Size Exclusion Support | MWCO | Cationic Moiety |
|---|---|---|---|
| Resin A | Resin 1 | 2 kDa | 150 mg/mL PDA |
| Resin B | Resin 2 | 7 kDa | 150 mg/mL PDA |
| Resin C | Resin 2 | 7 kDa | 150 mg/mL DEED |
| Resin D | Resin 3 | 40 kDa | 150 mg/mL PDA |
| Resin E | Resin 4 | 30 kDa | 150 mg/mL PDA |
| Resin F | Resin 5 | 45 kDa | 150 mg/mL PDA |
| Resin G | Resin 6 | 50 kDa | 150 mg/mL PDA |
| Resin H | Resin 7 | 80 kDa | 75 mg/mL PDA |
| Resin I | Resin 8 | 90 kDa | 75 mg/mL PDA |
| Resin J | Resin 5 | 45 kDa | 150 mg/mL PDA + |
| | | | 50 mg/mL Dextran |
| Resin K | Resin 5 | 45 kDa | 75 mg/mL PDA |
| Resin L | Resin 5 | 45 kDa | 50 mg/mL PDA |

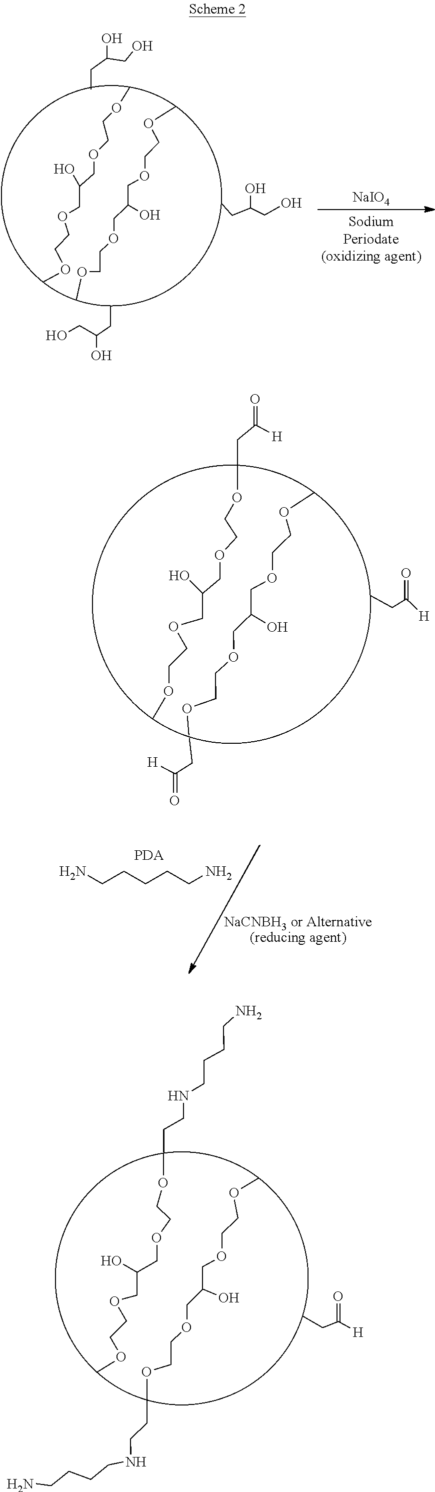

Scheme 2 involves the following steps: (1) 100 mL's of size exclusion base matrix resin were prepared; (2) 2.3 grams of sodium metaperiodate were dissolved in water to prepare 100 mL's of 23 mg sodium metaperiodate/mL of resin; (3) adding 100 mL's of 23 mg sodium metaperiodate mL of resin to the 100 mL's of size exclusion matrix resin bed; (4) the reaction was allowed to proceed for 4 hours at room temperature with constant overhead stirring, thereby oxidizing vicinal diols present in the HEC to aldehyde groups; (5) preparing 100 mL volume of 50-150 mg/mL concentrations of the cationic moiety such as, but not limited to, PDA, PEI, or DEED in PBS and adjusting the pH of solution to be in the range of 8.0-8.5; (6) adding the prepared reagent to the resin slurry, whereby the amines provided by the reagents react with the aldehyde groups formed in the periodate oxidizing (step 4) and adding 10 mg sodium cyanoborohydride/mL of the resin to perform reductive amination; (7) allowing the reaction to proceed for 8-12 hours at room temperature with constant overhead stirring; and (8) washing the resin with 2 bed volumes water, 2 bed volumes NaCl, and 2 bed volumes water.

Method for Evaluating the Resin on BSA-IgG Mixture: (1) Spin 562 μL, of resin bed at 3000×G for 1 minute to remove the storage solution; (2) the resin was equilibrated with buffer (3×300 μL); (3) spin at 3000×G for 1 minute to remove the buffer; (4) 100 μL of BSA-Rabbit IgG mixture (BSA 10 mg/mL and IgG 1 mg/mL) to the resin; (5) spin at 3000×G for 2 minutes to collect flow-through, which contained unbound IgG and unbound BSA); (6) prepare a 1:10 dilution of the flow-through and start; add 10 μL/well to a 4-20% Tris Glycine gel; (7) run gel at 200 V for 45 minutes-50 minutes; (8) stain/destain the gel using Pierce Power stainer.

### Example 2

Resin MWCO Effect on BSA Removal and Antibody Recovery: In this example, the MWCO effect of the Resin A embodiment, the Resin B embodiment, the Resin D embodiment, the Resin E embodiment, the Resin F embodiment of Table 2 were compared to determine the effect of increasing the MWCO on the removal of BSA from a BSA (2 mg/mL) and IgG (2 mg/mL) mixture.

FIG. 9 is an image of a gel showing the removal of BSA from the BSA/IgG described above and in Table 2. As shown by FIG. 9, the BSA removal property, which is indicated by lower band disappearance in the gel, increased in the following order respectively: the Resin F embodiment (45 kDa MWCO)>the Resin D embodiment (40 kDa MWCO)>the Resin E embodiment (30 kDa MWCO)>the Resin B embodiment (7 kDa MWCO)>the Resin A embodiment (2 kDa MWCO). Thus, the MWCO in addition to the PDA modified embodiments, had a significant effect on removing BSA from a sample.

### Example 3

Effect of PDA Modification and Equilibration Buffer: In this example, the Resin 5 embodiment of Table 1, the Resin L embodiment of Table 2, the Resin F embodiment of Table 2, the Resin D embodiment of Table 2, wherein different buffers were used for equilibration as shown in Table 3 and were evaluated for their ability to remove BSA and recovery IgG from a BSA-IgG mixture.

Melon Gel Purification Buffer (MGPB): Low phosphate concentration buffer with no sodium chloride. Without being bound by this theory, the low negative charge of this buffer was selected to increase the BSA binding capacity to resin embodiments having a positive charge.

Tris Buffer: A positively charged buffer with no negative charge. Without being bound by this theory, the absence of a negative charge will allow for the BSA binding capacity to increase, similar to MGPB; however, also repel positively charged amine groups on IgG and thereby increase the IgG recovery.

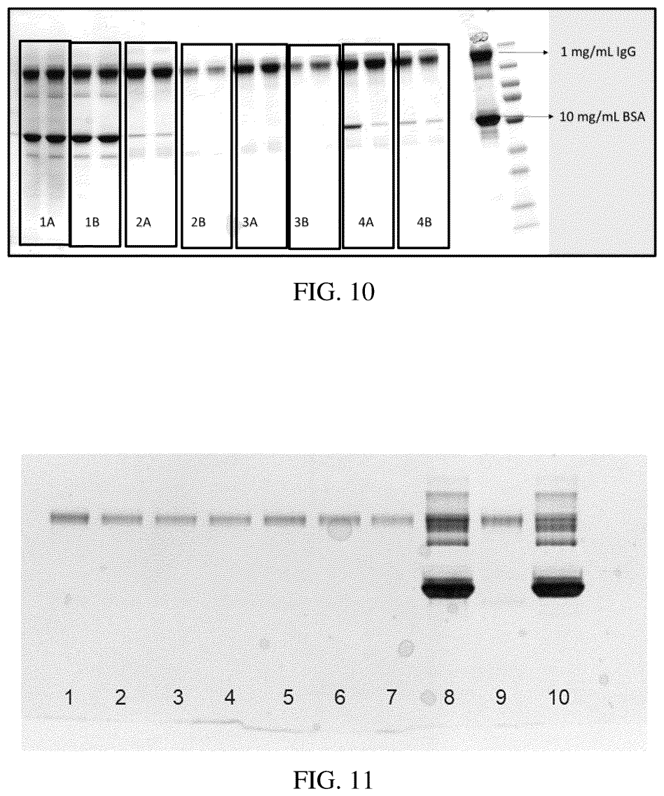

FIG. 10 is an image of a gel showing the BSA removal and IgG recovery according to Table 3.

TABLE 3

| Lane # | Resin | MWCO | PDA (mg/mL) | Buffer |
|---|---|---|---|---|
| 1A | Resin 5 | 45 kDa | 0 | 50 mM Tris |
| 1B | Resin 5 | 45 kDa | 0 | MGPB |
| 2A | Resin L | 45 kDa | 50 | 50 mM Tris |
| 2B | Resin L | 45 kDa | 50 | MGPB |
| 3A | Resin F | 45 kDa | 150 | 50 mM Tris |
| 3B | Resin F | 45 kDa | 150 | MGPB |
| 4A | Resin D | 40 kDa | 150 | 50 mM Tris |
| 4B | Resin D | 40 kDa | 150 | MGPB |

As shown in FIG. 10 and Table 3, Lane 1A and Lane 1B show no BSA removal, however, the PDA modified resin embodiments removed more BSA from the BSA-IgG mixture. Moreover, the concentration of PDA (50 mg/mL versus 150 mg/mL) had little effect on BSA removal and the 40 kDa MWCO resin embodiments in Lanes 4A and 4B showed a lower capacity to remove BSA than the 45 kDa MWCO resin embodiments in Lanes 3A and 3B. Furthermore, the buffer used for equilibration demonstrated that the resin embodiments equilibrated with MGPB exhibited a lower IgG recovery than resin embodiments equilibrated with 50 mM Tris.

Example 4

In this example, the ability of the Resin G embodiment according to Table using different equilibration buffers to remove BSA and recovery IgG from a sample comprising a mixture of BSA (10 mg/mL) and IgG (1 mg/mL) was compared.

FIG. 11 is an image of a gel showing the removal of BSA and IgG recovery for a sample comprising a mixture of BSA (10 mg/mL) and IgG (1 mg/mL) by the Resin G embodiment (as described in Table 1, provided herein) equilibrated with 50 mM Tris pH 7 (Lane 1), 50 mM TEAB pH 5 (Lane 2), 50 mM TEAB pH 7 (Lane 3), 50 mM sodium acetate pH 5 (Lane 4), 50 mM sodium acetate pH 7 (Lane 5), 50 mM HEPES pH 5 (Lane 6), 50 mM HEPES pH 7 (Lane 7), 1 mg/mL rabbit IgG/10 mg /mL BSA (Lane 8), 1 mg/mL rabbit IgG, (Lane 9), 10 mg/mL BSA.

This example demonstrates desirable BSA removal and IgG recovery with different equilibration buffers having a pH from 5 to 7. Moreover, equilibration buffers having a neutral charge, such as sodium acetate, exhibited desirable BSA removal and IgG recovery.

### Example 5

In this example, the BSA binding capacity (300 μL of 10 mg/mL) and the IgG recovery (2 mg/mL) of the Resin 5 embodiment of Table 1 modified with 150 mg/mL PEI was compared to the Resin 3 embodiment of Table 1 modified with 150 mg/mL PEI, Affi-Gel® Blue Gel (Bio-Rad), and the Melon™ Gel IgG Purification Kit (Thermo Scientific™) in a sample comprising 300 μL of 10 mg/mL.

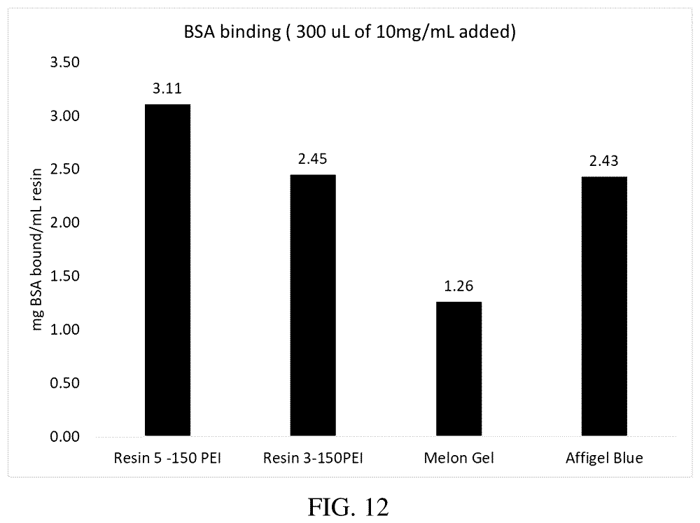

FIG. 12 is a bar graph showing the mg BSA bound/mL resin. As shown in FIG. 12, the Resin 5 embodiment (as described in Table 1, provided herein) had a binding capacity of 3.11 mg BSA bound/mL resin, the Resin 3 embodiment (as described in Table 1, provided herein) had a binding capacity of 2.45 mg BSA bound/mL resin, the Melon™ Gel IgG Purification Kit had a binding capacity of 1.26 mg BSA bound/mL resin, and the Affi-Ge® Blue Gel (Bio-Rad) had a binding capacity of 2.43 mg BSA bound/mL resin.

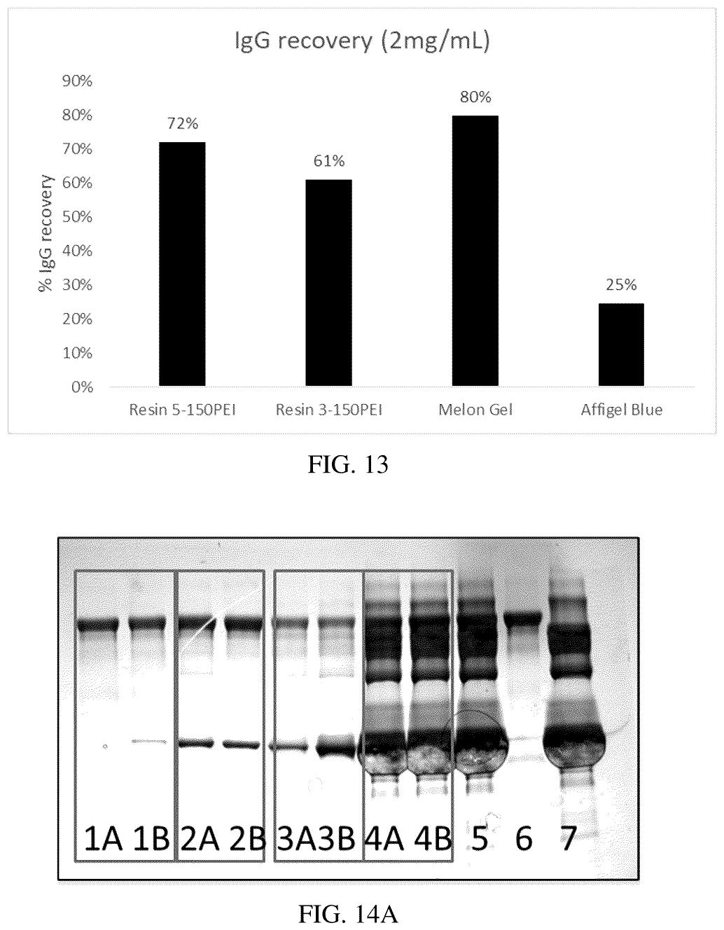

FIG. 13 is a bar graph showing the IgG recovery (2 mg/mL). Accordingly, the Resin 5 embodiment (as described in Table 1, provided herein) exhibited a 72% volume recovery, the Resin 3 embodiment (as described in Table 1, provided herein) exhibited a 61% volume recovery, the Melon™ Gel IgG Purification Kit exhibited an 80% volume recovery, and the Affi-Gel® Blue Gel (Bio-Rad) exhibited a 25% volume recovery.

Therefore, this example demonstrates that desirable BSA binding capacity was achieved when PEI was used as the modification reagent, had a comparable BSA binding capacity to the Affi-Gel® Blue Gel (Bio-Rad), and comparable IgG recovery to the Melon™ Gel IgG Purification Kit.

### Example 6

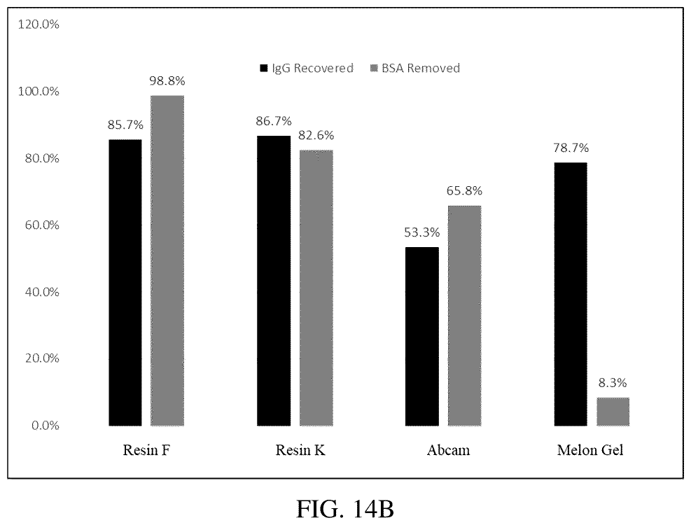

In this example, the Resin 5 embodiments of Table 1 were modified with different concentrations of PDA to produce the Resin F embodiment and the Resin K embodiment as described in Table 2, which were compared to commercially available Abcam BSA Removal Kit and Melon™ Gel IgG Purification Kit (Thermo Scientific™) for their ability to remove BSA and recovery IgG from a sample comprising a BSA-IgG mixture. Table 4 presents the data of FIG. 14A, which is an image of a gel showing the BSA removal capacity and IgG recovery capacity of the Resin 5 embodiments modified with different concentrations of PDA to produce the Resin F embodiment and the Resin K embodiment versus Abcam BSA Removal Kit and Melon™ Gel IgG Purification Kit (Thermo Scientific™). As shown in FIG. 14A, Lane 5 is the BSA 10 mg/mL and GAR 1 mg/mL mixture; Lane 6 is the 1 mg/mL GAR and Lane 7 is the BSA 10 mg/mL; and all lanes were normalized and samples were loaded at 10 μL/well on the gel.

TABLE 4

| Lane # | Resin | MWCO | PDA (mg/mL) |
|---|---|---|---|
| 1A | Resin F | 45 kDa | 150 |
| 1B | Resin F | 45 kDa | 150 |
| 2A | Resin K | 45 kDa | 75 |
| 2B | Resin K | 45 kDa | 75 |
| 3A | Abcam BSA Removal Kit | N/A | N/A |
| 3B | Abcam BSA Removal Kit | N/A | N/A |
| 4A | Melon ™ Gel IgG Purification Kit | N/A | N/A |
| 4B | Melon ™ Gel IgG Purification Kit | N/A | N/A |

FIG. 14A shows superior BSA removal IgG recovery properties in Lane 1 and Lane 2 when compared to commercially available Abcam BSA Removal Kit and the Melon™ Gel IgG Purification Kit (Thermo Scientific™) in Lane 3 and Lane 4 respectively.

FIG. 14B is the is a bar graph obtained from quantitating the bands from FIG. 14A using IBright image analysis software. Accordingly, the Resin F embodiment (Resin 5 embodiment of Table 1 modified with 150 mg/mL PDA) had a 85.7% IgG recovery and a 98.8% BSA removal; Resin K (Resin 5 embodiment of Table 1 modified with 75 mg/mL PDA) showed an 86.7% IgG recovery and an 82.6% BSA removal; Abcam BSA Removal Kit had a 53.3% IgG recovery and a 65.78% BSA removal; and Melon™ Gel IgG Purification Kit (Thermo Scientific™) had a 78.7% IgG recovery and a 8.3% BSA removal. Therefore, the Resin F embodiment and the Resin K embodiment achieved a higher IgG recovery and BSA removal than the Abcam BSA Removal Kit and the Melon™ Gel IgG Purification Kit (Thermo Scientific™).

### Example 7

Increasing MWCO: In this example, higher MWCO resins having a MWCO of 45,000 Da were produced along with PDA modification and were measured for their abilities to remove BSA removal and recover IgG from a sample comprising BSA-IgG. Higher MWCO was achieved in the Resin 5 embodiment, Resin 6 embodiment, Resin 7 embodiment, and Resin 8 embodiment were prepared according to Table 1.

FIG. 15 is a bar graph showing recovery percentage at 42,000 Da, 67,000 Da, 80,000 Da, and 150,000 Da for the Resin 5 embodiment, the Resin 6 embodiment, the Resin 7 embodiment, and the Resin 8 embodiment. Accordingly, the Resin 5 embodiment exhibited an 86% at 42,000 Da, 94% recovery at 67,000 Da, 92% recovery at 80,000 Da, and 94% recovery at 150,000 Da. The Resin 6 embodiment exhibited an 82% recovery at 42,000 Da, 84% recovery at 67,000 Da, 90% recovery at 80,000 Da, and 92% recovery at 150,000 Da. The Resin 7 embodiment exhibited a 66% recovery at 42,000 Da, 76% recovery at 67,000 Da, 84% recovery at 80,000 Da, and 86% recovery at 150,000 Da. The Resin 8 embodiment exhibited a 58% recovery at 42,000 Da, 75% recovery at 67,000 Da, 75% recovery at 80,000 Da, and 83% recovery at 150,000 Da.

This example demonstrates that by decreasing the amount of HEC produced resins with a 50 K MWCO, 80 K MWCO, and a 90 K MWCO (see Table 1).

### Example 8

In this example, the Resin F embodiment, the Resin G embodiment, the Resin H embodiment, Resin I embodiment, and Resin J were produced according to Table 2 and were tested for their ability to remove BSA and recover GAR from a sample comprising a BSA-GAR mixture and compared to commercially available Melon™ Gel IgG Purification Kit (Thermo Scientific™), Affi-Gel® Blue Gel (Bio-Rad), and Abcam BSA Removal Kit.

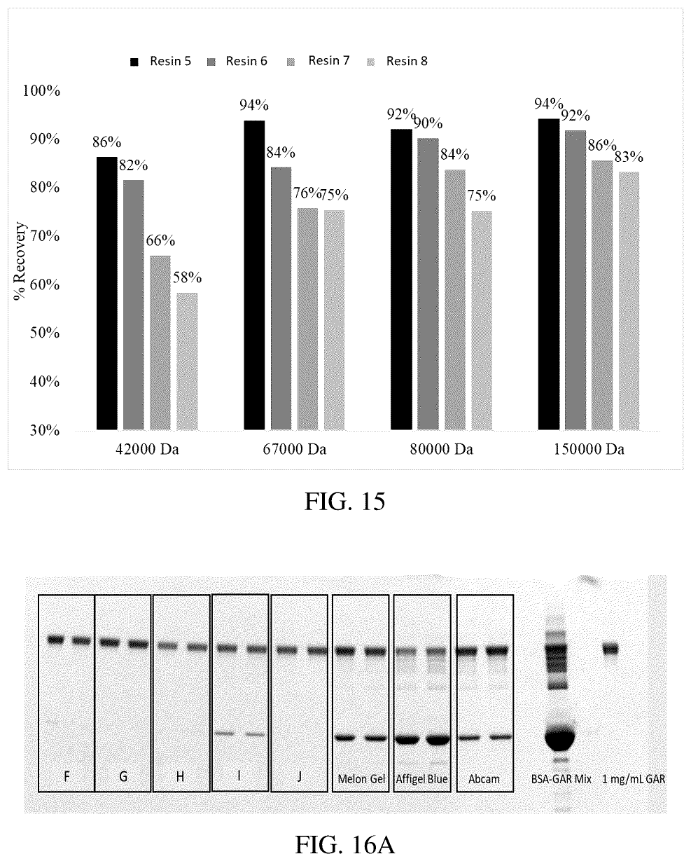

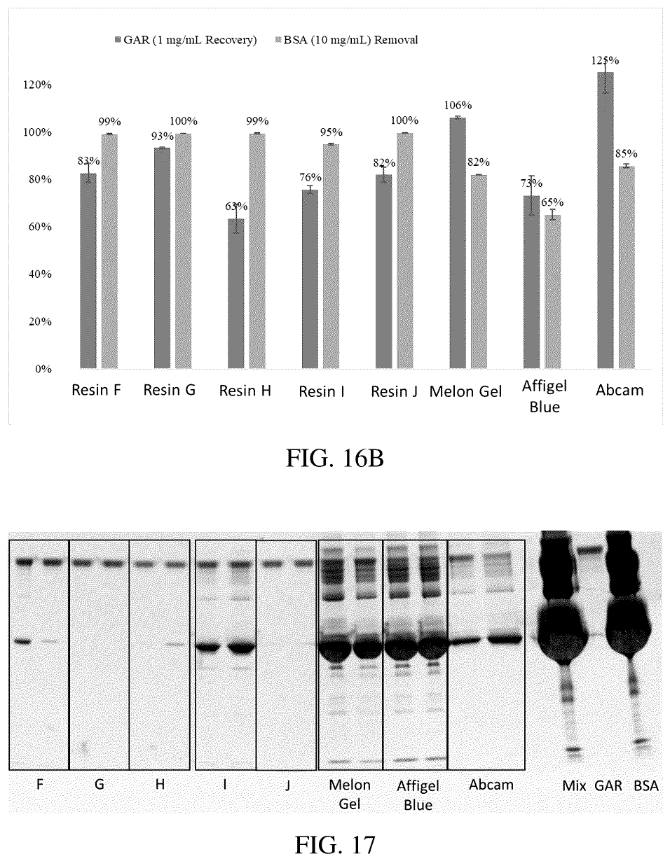

FIG. 16A is an image of a gel showing the BSA removal and antibody recovery and Table 5 is a key of FIG. 16A.

TABLE 5

| Lane: | |
|---|---|
| F | Resin F |
| G | Resin G |
| H | Resin H |
| I | Resin I |
| J | Resin J |
| Melon Gel | Melon ™ Gel IgG Purification Kit |
| Affigel Blue | Affi-Gel ® Blue Gel |
| Abcam | Abcam BSA Removal Kit |

As shown in FIG. 16A, the Resin F embodiment (45 K MWCO) and Resin G embodiment (50 K MWCO) showed the most BSA removal and IgG recovery from the sample comprising the BSA (10 mg/mL) and GAR IgG (1 mg/mL) mixture when compared to the Melo™ Gel IgG Purification Kit (Thermo Scientific™), Affi-Gel® (Bio-Rad) and Abcam BSA Removal Kit. The Resin H embodiment (80 K MWCO) and the Resin I embodiment (90 K MWCO), which were modified with 75 mg/mL PDA performed better in removing BSA and recovering IgG when compared to the Melon™ Gel IgG Purification Kit (Thermo Scientific™), Affi-Gel® Blue Gel (Bio-Rad), and Abcam BSA Removal Kit; however, did not perform as well in recovering IgG than the Resin F embodiment and the Resin G embodiment. Resin J was produced by reacting the Resin 6 embodiment of Table 1 with PDA and then was further reacted the open amine end of PDA with dextran having a molecular weight of 1,000,000; however, this did not improve the performance in comparison to Resin F and G.

FIG. 16B is a bar graph further demonstrating the GAR (1 mg/mL) recovery and BSA (10 mg/mL) removal. As shown in FIG. 16B, the Resin F embodiment showed a 83% GAR recovery and a 99% BSA removal; the Resin G embodiment showed a 93% GAR recovery and a 100% BSA removal; the Resin H embodiment showed a 63% GAR recovery and a 99% BSA removal; the Resin I embodiment showed a 76% GAR recovery and a 95% BSA removal; the Resin J embodiment showed a 82% GAR recovery and a 100% BSA removal; Melon™ Gel IgG Purification Kit (Thermo Scientific™) showed a 106% GAR recovery and a 82% BSA removal; Affi-Gel® Blue Gel (Bio-Rad) showed a 73% GAR recovery and a 65% BSA removal; and Abcam BSA Removal Kit showed a 125% GAR recovery and a 85% BSA removal. Moreover, artificially high IgG recoveries shown by the Abcam BSA Removal Kit and Melon™ Gel IgG Purification Kit (Thermo Scientific™) data can be explained because poor BSA removal runs a smear in the gel, which leads to band overlap with the IgG. Therefore, this example demonstrated a higher BSA removal and superior IgG recovery for the Resin F embodiment, Resin G embodiment, and Resin J embodiment when compared to commercially available resins and the Resin H embodiment and the Resin I embodiment.

### Example 9

In this example, the Resin F embodiment, Resin G embodiment, Resin H embodiment, Resin I embodiment, and Resin J embodiment were produced according to Table 2 and compared to Melon™ Gel IgG Purification Kit (Thermo Scientific™), Affi-Gel® Blue Gel (Bio-Rad), and Abcam BSA Removal Kit for their ability to remove BSA and IgG from a sample comprising a mixture of BSA (10 mg/mL) and GAR IgG (0.1 mg/mL).

FIG. 17 is an image of a gel showing the BSA removal and IgG recovery and Table 6 is a key of FIG. 17.

TABLE 6

| Lane: | |
|---|---|
| F | Resin F |
| G | Resin G |
| H | Resin H |
| I | Resin I |
| J | Resin J |
| Melon Gel | Melon ™ Gel IgG Purification Kit |
| Affigel Blue | Affi-Gel ® Blue Gel |
| Abcam | Abcam BSA Removal Kit |

As shown in FIG. 17, the Resin F embodiment and the Resin G embodiment showed the best BSA removal and IgG recovery from the mixture comprising BSA (10 mg/mL) and GAR IgG (0.1 mg/mL), when compared to Melon™ Gel IgG Purification Kit (Thermo Scientific™), Affi-Gel® Blue Gel (Bio-Rad), and Abcam BSA Removal Kit. Furthermore, Resin H performed better than Melon™ Gel IgG Purification Kit (Thermo Scientific™), Affi-Gel® Blue Gel (Bio-Rad), and Abcam BSA Removal Kit; however, did not perform as well in recovering IgG than the Resin F embodiment and the Resin G embodiment. The Resin I embodiment showed a lower ability to remove BSA when the sample comprised a 0.1 mg/mL GAR and 10 mg/mL BSA mixture. The Resin J embodiment did not perform better than the Resin F embodiment or the Resin G embodiment. The Melon™ Gel IgG Purification Kit (Thermo Scientific™), Affi-Gel® Blue Gel (Bio-Rad), and Abcam BSA Removal Kit performed poorly in removing BSA and recovering GAR IgG in sample comprising a low GAR-BSA mixture (0.1 mg/mL GAR and 10 mg/mL BSA. Lane 11 shows the BSA (10 mg/mL) only, which shows the smear obtained by running only BSA.

### Example 10

In this example, a mixture of GAR (1 mg/mL) and BSA (10 mg/mL) was passed and treated through the Resin F embodiment according to Table 2, Melon™ Gel IgG Purification Kit (Thermo Scientific™), Affi-Gel® Blue Gel (Bio-Rad), and Abcam BSA Removal Kit. The resulting flow-through was then labeled with NHS DyLight™ 488 (Thermo Scientific™) and the free dye was cleaned up using Pierce™ Dye and Biotin Removal Resin.

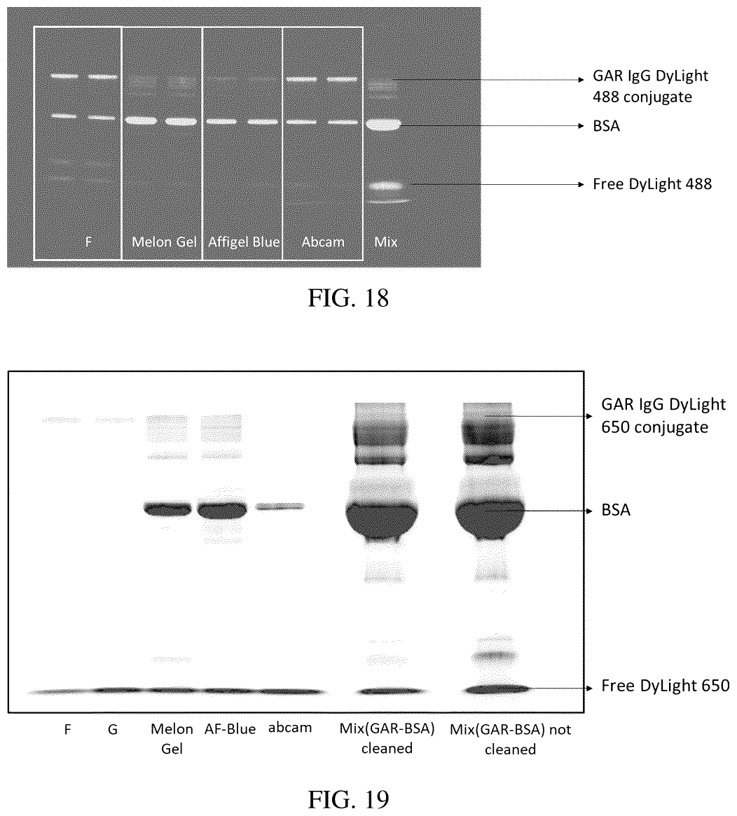

FIG. 18 shows the fluorescent dye labeling to BSA removed from the antibody before the labeling reaction and Table 7 shows a key corresponding to FIG. 18.

TABLE 7

| Lane: | |
|---|---|
| F | Resin F |
| Melon Gel | Melon ™ Gel IgG Purification Kit |
| Affigel Blue | Affi-Gel ® Blue Gel |
| Abcam | Abcam BSA Removal Kit |

FIG. 18 shows the efficiency of labeling to the GAR when BSA was removed as demonstrated by the Resin F embodiment versus the Melon™ Gel IgG Purification Kit (Thermo Scientific™) and Affi-Gel® Blue Gel (Bio-Rad) in which the labeling was comprised due to the unremoved BSA. Abcam BSA Removal Kit demonstrated comparable results with the Resin F embodiment.

FIG. 19 is an image of the fluorescent dye labeling to BSA removed from the antibody before labeling the reaction. A mixture of GAR (0.1 mg/mL) and BSA (10 mg/mL) was passed through the Resin F embodiment according to Table 2, the Resin G embodiment according to Table 2, Melon™ Gel IgG Purification Kit (Thermo Scientific™) , Affi-Gel® Blue Gel (Bio-Rad), and BSA Removal Kit. The resulting flow-through was then labeled with NHS DyLight™ 650 (Thermo Scientific™) and the free dye cleaned up using Pierce™ Dye and Biotin Removal Resin. Table 8 is a key of FIG. 19.

TABLE 8

| Lane | |
|---|---|
| F | Resin F |
| G | Resin G |
| Melon Gel | Melon ™ Gel IgG Purification Kit |
| AF-blue | Affi-Gel ® Blue Gel |
| Abcam | Abcam BSA Removal Kit |
| Mix (GAR-BSA) cleaned | Mixture (GAR-BSA) Cleaned |
| Mix (GAR-BSA) cleaned | Mixture (GAR-BSA) not cleaned |

FIG. 19 demonstrates the efficiency of labeling to the GAR when BSA was removed as demonstrated by the Resin F embodiment and the Resin G embodiment when compared to Melon™ Gel IgG Purification Kit, Affi-Gel® Blue Gel (Bio-Rad), Abcam BSA Removal Kit in which the labeling efficiency was comprised due to unremoved BSA. Thus, FIG. 19 indicates that even at low antibody concentrations such as 0.1 mg/mL, the Resin F embodiment and the Resin G embodiment were able to recover the antibody after the BSA was cleaned up and were able to successfully dye label it.

### Example 11

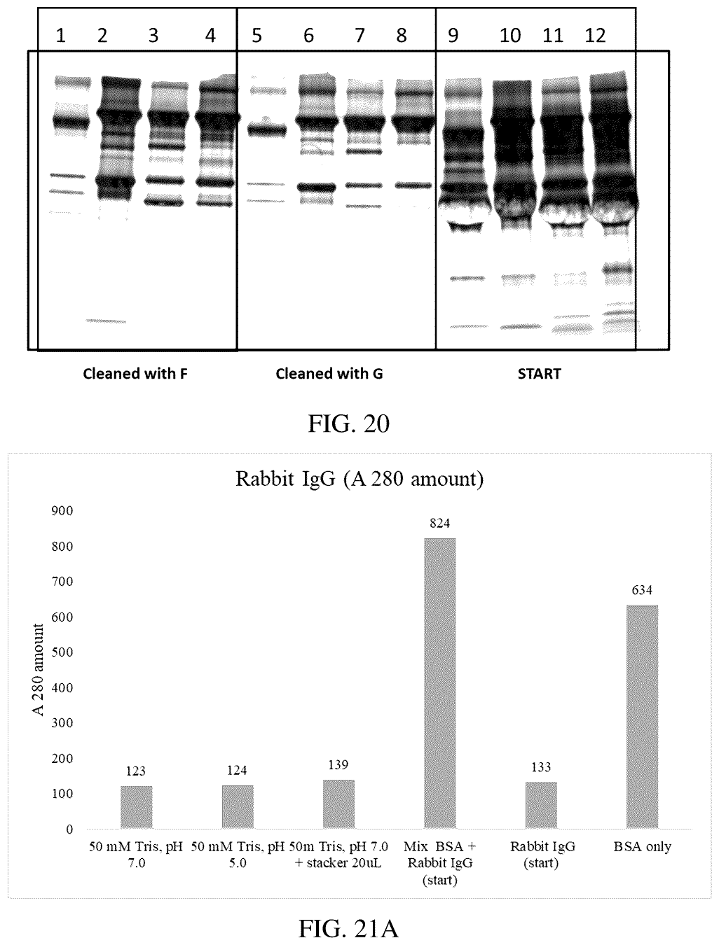

Removal of Serum from Serum Samples: In this example, the Resin F embodiment according to Table 2 and the Resin G embodiment were used to clean up rabbit serum, mouse serum, human plasma, and human serum. FIG. 20 is an image of a gel showing the results of the Resin F embodiment and the Resin G embodiment versus the start mixture where Lanes 1, 5, and 9 comprise the rabbit serum, Lanes 2, 6, 10 comprise the mouse serum, Lanes 3, 7, 11 comprise the human plasma, and Lanes 4, 8, and 12 comprise the human serum; and Table 9 shows the data of FIG. 20.

TABLE 9

| Lane # | Rabbit Serum | Lane # | Human Plasma |
|---|---|---|---|
| 9 | Start | 11 | Start |
| 1 | Cleaned with the Resin F embodiment | 3 | Cleaned with the Resin F embodiment |
| 5 | Cleaned with the Resin G embodiment | 7 | Cleaned with the Resin G embodiment |
| Lane # | Mouse Serum | Lane # | Human Serum |
| 10 | Start | 12 | Start |
| 2 | Cleaned with the Resin F embodiment | 4 | Cleaned with the Resin F embodiment |
| 6 | Cleaned with the Resin G embodiment | 8 | Cleaned with the Resin G embodiment |

As shown in FIG. 20, the Resin F embodiment and the Resin G embodiment demonstrated an excellent ability to remove albumin and a good ability in recovering IgG from different serum species.

### Example 12

Effect of Equilibration Buffer pH: In this example, the effect of the equilibration buffer Tris 50 mM on the ability to remove BSA and recover IgG on the Resin G embodiment according to Table 2. The starting amount and the flow-through amounts were measured at A 280 using a nanodrop. The A 280 amount was calculated by measuring the measuring the A 280 on the nanodrop and taking the volume of the sample added and recovered into account. A 280 measurement of BSA at 10 mg/mL was determined using the Nanodrop as the control.

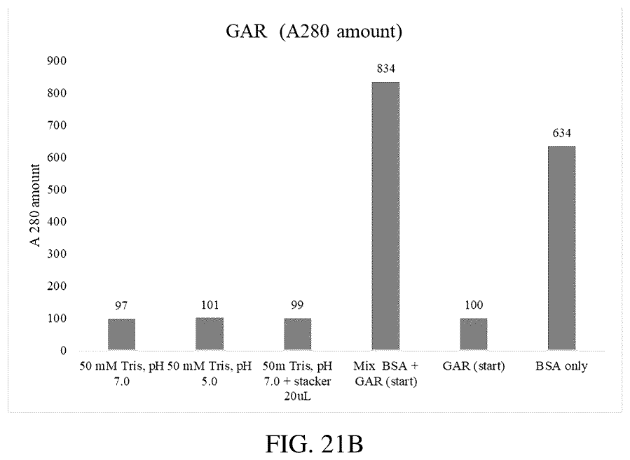

FIG. 21A is a bar graph showing the A 280 amount of the rabbit IgG A 280 amount. As shown in FIG. 21A, the Resin G embodiment equilibrated with 50 mM Tris (pH 7.0) had a 123 A 280 amount; the Resin G embodiment equilibrated with 50 mM Tris (pH 5.0) had a 124 A 280 amount; the resin G embodiment equilibrated with 50 m Tris (pH 7.0 +stacker 20 μL) had a 139 A 280 amount; the BSA-rabbit IgG start mixture had a 824 A 280 amount; the Rabbit IgG start had a 133 A 280 amount; and the BSA only had a 634 A 280 amount.

FIG. 21B is a bar graph showing the A 280 amount of the GAR. As shown in FIG. 21B, the Resin G embodiment equilibrated with 50 mM Tris (pH 7.0) had a 97 A 280 amount; the Resin G embodiment equilibrated with 50 mM Tris (pH 5.0) had a 101 A 280 amount; the resin G embodiment equilibrated with 50 m Tris (pH 7.0 +stacker 20 μL) had a 99 A 280 amount; the BSA-GAR start mixture had a 834 A 280 amount; the GAR start had a 100 A 280 amount; and the BSA only had a 634 A 280 amount.

This example demonstrates the flow-through amount of A 280 was similar to A 280 amount from the GAR and rabbit IgG, which indicated the desired recovery of antibody after cleaning up with the Resin G embodiment.

### Example 13

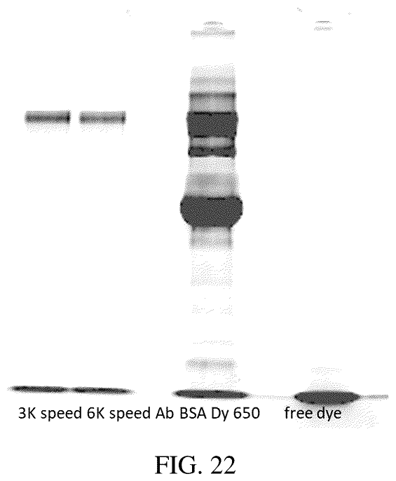

Labeling of Primary Antibody: In this example, the primary antibody GAPDH was labeled with a fluorescent dye after the removal of BSA from the BSA-antibody mixture. FIG. 22 is an image of a gel showing the removal of BSA from the primary antibody GAPDH before conjugating it with DyLight™ 650 (Thermo Scientific™) and Table 10 shows the data of FIG. 22.

TABLE 10

| Lane: | |
|---|---|
| 1 | Resin G spun at 3K speed |
| 2 | Resin G spun at 3k speed |
| 3 | Ab-BSA Dy650 |
| 4 | Free Dye |

The resin was spun at 3000×G and 6000×G. 1 mg/mL GAPDH was spiked with 10 mg/mL BSA and passed through the Resin G embodiment according to Table 2. The flow-through was collected and the resin was washed with 50 mM Tris to collect the GAPDH that may have been bound to the resin. The flow-throughs from the two elution's were collected, pooled, then conjugated with NHS DyLight™ 650 (Thermo Scientific™) and cleaned using Pierce™ Dye and Biotin Removal Resin. GAPDH with BSA was also labeled and Free Dy 650 were added as control lanes was also labeled.

This example demonstrates successful removal of BSA from primary antibody GAPDH before conjugating with DyLight™ 650 (Thermo Scientific™) and thus demonstrates complete removal of BSA for spin speeds at 3,000×G and 6,000×G.

### Example 14

In this example, the ability the Resin G embodiment according to Table 2 to remove BSA from primary antibody Calreticulin before conjugating it with DyLight™ 680 NHS (Thermo Scientific™) was tested. 1 mg/mL Calreticulin antibody was obtained with 1 mg/mL BSA added as a stabilizer. This antibody was passed through the Resin G embodiment and the flow-through was collected and the resin was washed three times with 50 mM Tris to collect the primary antibody that may have been bound to the resin. The flow-throughs were collected, pooled, concentrated, and then conjugated with DyLight™ 680 NHS (Thermo Scientific™) and cleaned using Pierce™ Dye and Biotin Removal Resin.

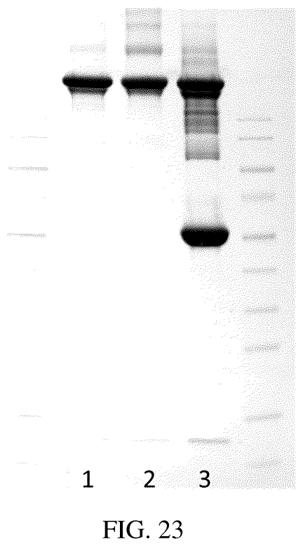

FIG. 23 is an image a gel obtained by loading the flow-throughs and stained using Coomassie stain using Pierce Power blotter. Table 11 shows the data of FIG. 23.

TABLE 11

| Lane # | |
|---|---|
| 1 | Calreticulin fee from BSA after passing through lane |
| 2 | Conjugated Calreticulin after BSA removal |
| 3 | Calreticulin received with added BSA as stabilizer |

As shown in FIG. 23, Lane 1 shows the Calreticulin that is free from BSA after passing through the Resin G embodiment. Lane 2 shows the Calreticulin that is conjugated to the DyLight™ 680 (Thermo Scientific™) after BSA is removed. Lane 3 is the Calreticulin as received with BSA added as a stabilizer. In view of this, this example demonstrates the successful removal of BSA from the primary antibody Calreticulin before conjugating it with DyLight™ 680 (Thermo Scientific™) by the Resin G embodiment.

### Example 15

Western Blot Application: In this example, Western Blot application using fluorescently labeled GAR after BSA cleanup was tested. 1 mg/mL goat anti-rabbit (GAR) was spiked with 10 mg/mL BSA. The Resin 7 embodiment according to Table 1 was modified with 150 mg/mL PDA was used to clean out the BSA. The GAR was then conjugated to DyLight™ 650 (Thermo Scientific™). GAR with BSA was also conjugated to DyLight™ 650 (Thermo Scientific™) as a control. HeLa Lysate was loaded at 10 μg, 2.5 μg, and 1.25 μg onto a gel, and then transferred to a nitrocellulose membrane. The membrane was blocked and then incubated with HSP 90 primary antibody. The membrane was washed and then incubated with HSP 90 primary antibody. The membrane was washed, then incubated with GAR Dy 650, washed and then scanned on IBright imager using the fluorescence mode. GAR conjugated with Dy 650 without BSA removal was used as a control for comparison.

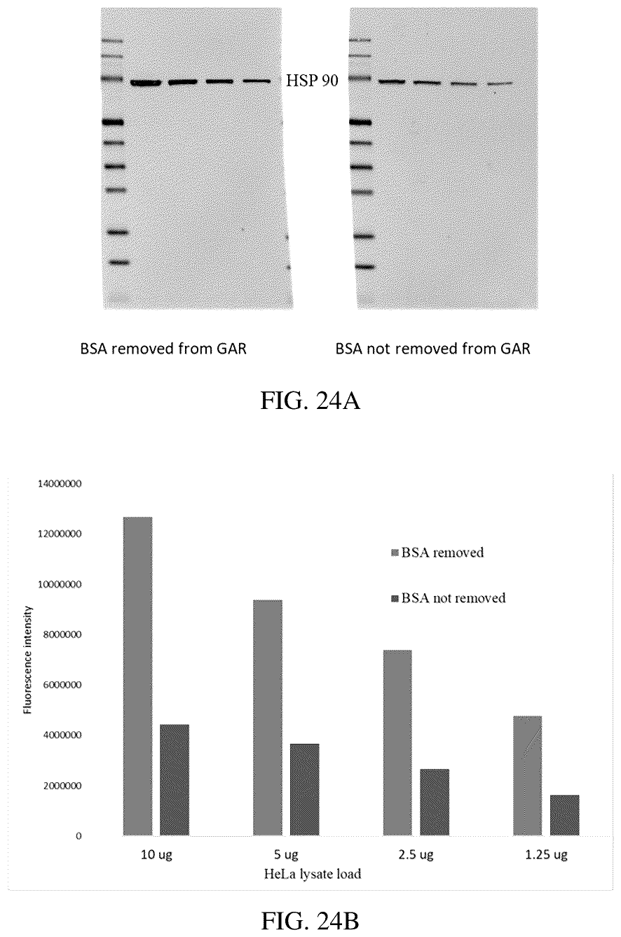

FIG. 24A is an image of a gel showing the BSA removed from GAR (left) and the BSA not removed from GAR (right). FIG. 24B is a bar graph showing the fluorescence intensity of the removed BSA and the unremoved BSA of the HeLa lysate load. As shown in FIG. 24B, HeLa lysate load for the BSA removed at 10 μg had a fluorescence intensity of 13,000,000 and the unremoved BSA had a fluorescence intensity of 4,000,000; HeLa lysate load for the BSA removed at 5 μg had a fluorescence intensity of 9,000,000 and the unremoved BSA had a fluorescence intensity of 3,800,000; HeLa lysate load for the BSA removed at 2.5 μg had a fluorescence intensity of 7,800,000 and the unremoved BSA had a fluorescence intensity of 3,800,000; and HeLa lysate load for the BSA removed at 1.25 μg had a fluorescence intensity of 4,100,000 and the unremoved BSA had a fluorescence intensity of 1,800,000.

This example demonstrates that from the blot and graph that BSA removed from the GAR before conjugating it to Dy 650 shows a much higher intensity on the blot as well as the quantitated graph than when BSA was not removed from GAR. Furthermore, the fluorescence intensity observed increased by a magnitude of three with GAR Dy650 with BSA removed. Thus, for low abundant targets in cell lysates or for poorly performing antibodies with BSA, performance can be enhanced by removing BSA with the Resin 7 embodiment modified with 150 mg/mL PDA.

While preferred embodiments of the present disclosure have been shown and described herein, it will be apparent to a person of ordinary skill in the art that such embodiments are provided by way of example only. Variations, changes, and substitutions to these disclosed embodiments will be apparent to a person of ordinary skill in the art without departing from the present disclosure. It should be understood that all such various alternatives to the embodiments described herein may be employed in practicing the present disclosure. The following claims define the scope of the disclosure.

1. A matrix, comprising:
a porous size exclusion support; and
at least one cationic moiety associated with the porous size exclusion support, wherein the matrix can separate one or more molecules in a sample by using a molecular weight of the one or more molecules, a charge of the one or more molecules, an isoelectric point (pI) of the one or more molecules, or a combination thereof.

2. The matrix of claim 1, wherein the porous size exclusion support has a molecular weight cut-off of greater than or equal to 40 kDa.

3. The matrix of claim 1, wherein the at least one cationic moiety is covalently bound to the porous size exclusion support.

4. The matrix of claim 1, wherein the at least one cationic moiety comprises an amine, a diamine, a polyamine, amine-containing heterocyclic compound, amine-containing aromatic compound, or an amine-containing polymer.

5. The matrix of claim 4, wherein the at least one cationic moiety is polyethylene imine, diaminopentane, O-(2-Aminopropyl)-O′-(2-methoxyethyl)polypropylene glycol, 1,2-diaminobenzene, 1,3-diaminobenzene, 1,4-diaminobenzene, or KN-diethylethylenediamine.

6. The matrix of claim 1, wherein the porous size exclusion support comprises hydroxyethyl cellulose.

7. The matrix of claim 6, wherein the porous size exclusion support is crosslinked with epichlorohydrin.

8. A system for separating one or more molecules in a sample using a molecular weight of the one more molecules, the system comprising:
a container comprising the matrix of claim 1; and
a receptacle located to receive flow from the container, wherein the system is configured for gravity flow operation, centrifugal force operation, positive pressure operation, negative pressure operation, vacuum operation, or combinations thereof; and wherein the container is a columnar container, a tube, a multi-well tube, a multi-well plate, or a multi-well filter plate.

9. A method of making a multimodal resin, comprising:
providing a porous size exclusion support comprising hydroxyethyl cellulose having a MWCO of greater than or equal to 40 kDa, the hydroxyethyl cellulose having at least one vicinal diol;
oxidizing the least one vicinal diol to an aldehyde; and
reacting the aldehyde with an amine group of a cationic moiety via reductive amination.

10. The method of claim 9, wherein the porous size exclusion support is produced using an amount of hydroxyethyl cellulose ranging from 50 grams of to 250 grams.

11. The method of claim 9, further comprising providing a crosslinker to crosslink the porous size exclusion support.

12. The method of claim 11, wherein the crosslinker is epichlorohydrin and is used in an amount ranging from 250 milliliters to 450 milliliters.

13. The method of claim 9, wherein the cationic moiety is polyethylene imine, diaminopentane, O-(2-Aminopropyl)-O′-(2-methoxyethyl)polypropylene glycol, 1,2-diaminobenzene, 1,3-diaminobenzene, 1,4-diaminobenzene, or N,N-diethylethylenediamine.

14. The method of claim 13, wherein the cationic moiety is used at a concentration ranging from 50 mg/mL to 175 mg/mL.

15. A method for separating one or more molecules in a sample, comprising:
providing the matrix of claim 1;
equilibrating the matrix with an equilibration buffer; and
applying the sample to the matrix to separate the one or more molecules by subjecting the matrix to gravity flow, a centrifugal force, a positive pressure, a negative pressure, a vacuum, or a combination thereof,
wherein the one or more molecules of the sample is excluded by the matrix and is collected as a flow-through, and wherein the one or molecules associates with the at least one cationic moiety.

16. The method of claim 15, wherein the one or more molecules are removed without a desalting step.

17. The method of claim 15, wherein the flow-through is collected in a receptacle located below the container.

18-26. (canceled)

27. A kit for separating a positively charged large molecule from one or more negatively charged small molecules in a sample, the kit comprising:
a porous size exclusion support having at least one cationic moiety associated therewith, wherein the cationic moiety can associate with and capture the at least one negatively charged small molecule: and
instructions for using the porous size exclusion support.

28. The kit according to claim 27, further comprising an equilibration buffer, a container housing the porous size exclusion support, a receptacle positioned to receive flow-through the support, or any combination thereof.

29. The kit of claim 28, wherein the equilibration buffer comprises Tris, triethylammonium bicarbonate, sodium acetate, HEPES, or borate.
